(19) United States
(12) Patent Application Publication
(10) Pub. No.: US 2021/0308459 A1
(43) Pub. Date: Oct. 7, 2021

(54) STIMULATION TO GUIDE PHYSICAL THERAPY

(71) Applicant: Highland Instruments, Inc., Somerville, MA (US)

(72) Inventors: Timothy Andrew Wagner, Somerville, MA (US); Laura Dipietro, Cambridge, MA (US); Uri Tzvi Eden, Somerville, MA (US)

(73) Assignee: Highland Instruments, Inc., Somerville, MA (US)

(21) Appl. No.: 17/221,051

(22) Filed: Apr. 2, 2021

Related U.S. Application Data
(63) Continuation of application No. 16/208,081, filed on Dec. 3, 2018, now abandoned.
Application No. 16/208,081 is a continuation of application No. 14/335,282, filed on Jul. 18, 2014, now abandoned.
(60) Provisional application No. 61/856,284, filed on Jul. 19, 2013.

(51) Int. Cl. A61N 1/36; A61N 7/00; A61N 1/04; A61N 2/00

(52) U.S. Cl. CPC A61N 1/36025; A61N 7/00; A61N 5/0622; A61N 2/006; A61N 1/0408

(57) ABSTRACT

The invention generally relates to methods for guiding physical therapy to a subject. In certain embodiments, methods of the invention involve providing stimulation to a subject's central nervous system to modulate one or more signals sent to or from a plurality of target regions of the subject. Methods of the invention further involve assessing the response of the plurality of target regions to the stimulation to determine if there is a differential response among the target regions to the stimulation, and providing focused physical therapy to at least one of the target regions based on the assessment of the response of the plurality of peripheral target regions to the stimulation.

### RELATED APPLICATION

The present application is a continuation of U.S. application Ser. No. 14/335,282, filed Jul. 18, 2014, which claims the benefit of and priority to U.S. provisional application Ser. No. 61/856,284, filed Jul. 19, 2013, the content of each of which is incorporated by reference herein in its entirety.

### GOVERNMENT SUPPORT

This invention was made with government support under 5R44NS080632 awarded by the National Institute of Neurological Disorders and Stroke (NINDS) of the National Institutes of Health (NIH). The government has certain rights in the invention.

### FIELD OF THE INVENTION

The invention generally relates to methods for guiding physical therapy to a subject.

### BACKGROUND

Neuromodulation is the control of nerve activity, and is usually implemented for the purpose of treating disease. Neuromodulation may be accomplished with surgical intervention, such as cutting an aberrant nerve tract. However, the semi-permanent nature of a surgical procedure leaves little room for later adjustment and optimization. Neuromodulation may also be accomplished with chemical agents or medications. Chemical agents or medications may be undesirable because, for example, many medications are difficult to deliver to specific anatomy, and because the titration (increasing or decreasing the dose of a medication) is a slow and imprecise way to achieve a desired effect on a specific target.

Neuromodulation may also be accomplished using energy-delivering devices. The stimulation may be applied invasively, e.g., by performing surgery to remove a portion of the skull and implanting electrodes in a specific location within brain tissue, or non-invasively, e.g., transcranial direct current stimulation (tDCS) and transcranial magnetic stimulation (TMS).

The stimulation may act to modulate the plasticity of tissue (e.g., long term potentiation and/or long term depression). Long-term potentiation (LTP) involves the process of establishing an association between the firing of two cells or groups of cells. For instance, if an axon of cell A is near enough to excite a cell B and repeatedly or persistently takes part in firing cell B, an increase in the strength of the chemical synapse between the cells takes place such that A's efficiency, as one of the cells firing B, is increased. LTP has been shown to last from minutes to several months. Conditions for establishing LTP are favorable when a pre-synaptic neuron and a post-synaptic neuron are both depolarized in a synchronous manner. An opposite effect, long-term depression (LTD), has also been established. LTD is the weakening of a neuronal synapse that lasts from hours to months.

### SUMMARY

The invention recognizes that target regions of a subject respond differently to the same stimulation, and such information can be used to guide focused physical therapy. Such a combination can be used to obtain improved results over stimulation alone when physical therapy is focused on a region that already is more responsive to the stimulation than other target regions. Alternatively, such a combination can be used to obtain improved results over stimulation alone when physical therapy is focused on a region that is less responsive to the stimulation than other target regions.

Aspects of the invention are accomplished by providing stimulation to a subject's central nervous system (e.g., brain and/or spinal cord) to modulate one or more signals sent to or from a plurality of target regions of the subject. Methods of the invention further involve assessing the response of the plurality of target regions to the stimulation to determine if there is a differential response among the target regions to the stimulation, and providing focused physical therapy to at least one of the target regions based on the assessment of the response of the plurality of target regions to the stimulation. In certain embodiments, the focused physical therapy is provided to one or more target regions that is more responsive to the stimulation than other target regions. In other embodiments, the focused physical therapy is provided to one or more target regions that is less responsive to the stimulation than other target regions. In certain cases, the stimulation may be altered in response the subject's response to the physical therapy.

Exemplary signals that are modulated include pain related signals, inflammatory related signals, motor control signals, motor signals, proprioceptive signals, position signals, cognitive signals, sensory signals, auditory signals, visual signals, visuo-spatial signals, vibration signals, temperature signals, metabolic function signals, physiological function signals, fatigue signals, and/or coordination signals. However, methods of the invention may modulate any type of signal sent to or from a target and are not limited to those exemplary signals. Generally, the signal will be processed in the subject's brain. However, the signal may be processed in other parts of the subject's body, e.g., the spinal cord. In some embodiments the signals may be processed in multiple parts of the body, such as in the brain and spinal cord. Furthermore, the signal (e.g., electrical signal) may be processed in other parts of the body, such as the target regions and/or regions that are connected to target regions, such as through neural connections and/or neural muscular junctions and/or vascular connections and/or skeletal connections and/or connective tissue connections and/or lymphatic pathways, and be a target of therapy (which in turn can have reciprocal connections to the regions of stimulation, and/or directly impact the state of the system to stimulation, through additional signals originating from the target regions). In certain embodiments, effects of the stimulation alter neural function past the duration of stimulation. Thus, the effects of the treatment last significantly longer than the period of treatment.

Methods of the invention may be used to guide therapy for any neurological condition, such as neurodegenerative conditions, neuro-traumatic conditions, arthritic conditions, and/or chronic pain syndromes. In certain embodiments, the subject has a degenerative condition, such as Parkinson's disease. In the case of Parkinson's disease, an example of implementation involves providing stimulation that results in modulation of target regions such as the subject's upper arms above the elbow, the subject's lower arms below the elbow, and the subject's hands. In certain embodiments, the hands are more responsive to the stimulation than either the upper arms or the lower arms. In those embodiments, the focused physical therapy may be provided to the hands. Alternatively, the focused physical therapy may be provided to the upper arms, the lower arms, or a combination thereof. In the case of Parkinson's disease, an example of implementation involves providing stimulation that results in modulation of target regions such as the subject's upper legs above the knee, the subject's lower legs below the knee, and the subject's ankles. In certain embodiments, the ankles are more responsive to the stimulation than either the upper legs or the lower legs. In those embodiments, the focused physical therapy may be provided to the ankles. Alternatively, the focused physical therapy may be provided to the upper legs, the lower legs, or a combination thereof.

Any type of stimulation known in the art may be used with methods of the invention, and the stimulation may be provided in any clinically acceptable manner. For example, the stimulation may be provided invasively or noninvasively. Preferably, the stimulation is provided in a noninvasive manner. For example, electrodes may be configured to be applied to the specified tissue, tissues, or adjacent tissues. As one alternative, the electric source may be implanted inside the specified tissue, tissues, or adjacent tissues. Furthermore, the method could make use of combination of methods (e.g., invasive and noninvasive, multiple stimulation methods).

Exemplary types of stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). Other exemplary types include implant methods such as deep brain stimulation (DBS), microstimulation, spinal cord stimulation (SCS), and vagal nerve stimulation (VNS). In other embodiments, the stimulation source may work in part through the alteration of the nervous tissue electromagnetic properties, where stimulation occurs from an electric source capable of generating an electric field across a region of tissue and a means for altering the permittivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

In other embodiments, the stimulation is provided by a combination of an electric field and a mechanical field. The electric field may be pulsed, time varying, pulsed a plurality of times with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

The stimulation may be applied to a structure or multiple structures within the brain or the nervous system. Exemplary structures include dorsal lateral prefrontal cortex, any component of the basal ganglia, nucleus accumbens, gastric nuclei, brainstem, thalamus, inferior colliculus, superior colliculus, periaqueductal gray, primary motor cortex, premotor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, cortical structures, and spinal cord.

In one exemplary embodiment, the electric field is applied broadly and mechanical field is focused on a specific brain structure or multiple structures for therapeutic purposes. The electric field may be applied broadly and the mechanical field may be focused on a structure or multiple structures, such as brain or nervous tissues including dorsal lateral prefrontal cortex, any component of the basal ganglia, nucleus accumbens, gastric nuclei, brainstem, thalamus, inferior colliculus, superior colliculus, periaqueductal gray, primary motor cortex, pre-motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, cortical structures, sub-cortical structures, and/or spinal cord. Other possible configurations include applying both the electrical field and the mechanical field in a broad manner; applying both the electric field and the mechanical field in a focused manner; or applying the electric field in a focused manner and the mechanical field in a broad manner.

Other aspects of the invention provide methods for improving gait of a subject suffering from a neurological condition. Those methods involve providing stimulation to a subject's central nervous system, and providing physical therapy to the subject, thereby improving gait of the subject. Other aspects of the invention provide methods for improving bradykinesia in a subject suffering from a neurological condition. Those methods involve providing stimulation to a subject's central nervous system, and providing physical therapy to the subject, thereby improving bradykinesia in the subject. Other aspects of the invention provide methods for improving clinical scales in a subject suffering from a neurological condition. Those methods involve providing stimulation to a subject's central nervous system, and providing physical therapy to the subject, thereby improving clinical scales in the subject (such as the Unified Parkinson's Disease Rating Scale (UPDRS) in Parkinson's Disease Patients). Methods of the invention may be used to guide therapy for any neurological condition, such as neurodegenerative conditions or neuro-traumatic conditions. In certain embodiments, the neurological condition is Parkinson's disease. In certain embodiments, the physical therapy is adjusted based on the subject's response to the stimulation.

Furthermore, stimulation can be provided to affect gene expression in patients (such as for example affecting the expression of specific gene's in Familial type's Parkinson's Disease), or focused physical therapy can be provided to affect gene expression in patients (such as for example affecting the expression of specific gene's in Familial type's Parkinson's Disease), or the two can be used in concert in the manner described herein with gene therapy (or stimulation can be used in concert with gene therapy in a tuned manner).

Furthermore, stimulation can be provided to affect cellular protective mechanisms and/or growth factors and/or apoptosis inducing factors in patients (such as for example affecting neuroprotective agents and/or growth factors in Parkinson's Disease), or focused physical therapy to affect cellular protective mechanisms and/or growth factors and/or apoptosis inducing factors in patients (such as for example affecting neuroprotective agents and/or growth factors in Parkinson's Disease), or the two can be used in concert in the manner described herein. Furthermore the stimulation and/or therapy can be used with stem cell therapy or cell and/or tissue transplantation in a tuned and/or untuned manner.

### BRIEF DESCRIPTION OF THE DRAWINGS

FIG. 1 is one embodiment of an apparatus for stimulating biological tissue constructed in accordance with the principles of the present disclosure.

FIG. 2 is an exemplary embodiment of an apparatus for stimulating biological tissue constructed in accordance with the principles of the present disclosure.

FIG. 3 is an exemplary embodiment of an apparatus for stimulating biological tissue implementing a chemical source for altering permittivity constructed in accordance with the principles of the present disclosure.

FIG. 4 is an exemplary embodiment of an apparatus for stimulating biological tissue implementing a radiation source for altering permittivity constructed in accordance with the principles of the present disclosure.

FIG. 5 is another exemplary embodiment of an apparatus for stimulating biological tissue implementing an optical beam for altering permittivity constructed in accordance with the principles of the present disclosure.

FIG. 6 is a graph showing SCOPA-cog changes relative to baseline (as change in SCOPA score). Note bars depict 1 standard error

FIG. 7 is a graph showing 4-Choice reaction time improvements relative to baseline.

FIG. 8 is a graph showing Working Memory (as improvement in letter count) relative to baseline.

FIG. 9 is a graph showing Change in VAS Anxiety relative to baseline.

FIG. 10 is a graph showing Change in VAS Depression relative to baseline.

FIG. 11 is a graph showing Change in VAS Stress relative to baseline.

FIG. 12 is a graph showing Change in VAS Sleep relative to baseline.

FIG. 13 is a graph showing Change in VAS Depression relative to baseline as function of visit.

FIG. 14 is a graph showing Change in VAS Stress relative to baseline as function of visit.

FIG. 15 is a graph showing GAIT improvement.

FIG. 16 is a graph showing Walking Time Improvement.

FIG. 17 is a graph showing UPDRS Change.

FIG. 18 is a graph showing Bradykinesia changes.

FIG. 19 is a graph showing Bradykinesia Task Time Improvement.

FIG. 20 is a graph showing change in VAS pain scores during and following treatment.

### DETAILED DESCRIPTION

The invention generally relates to methods for guiding physical therapy to a subject. In certain aspects, methods of the invention involve providing stimulation to a subject's central nervous system to modulate one or more signals sent to or from a plurality of target regions of the subject, assessing the response of the plurality of target regions to the stimulation to determine if there is a differential response among the target regions to the stimulation, and providing focused physical therapy to at least one of the target regions based on results of the assessing step. Aspects of the invention are based on the data herein that show that target regions within a subject differentially respond to stimulation provided to the central nervous system. The differential response can be used to guide the physical therapy. For example, the therapy can be guided to focus on target regions that respond better to the stimulation than other regions of the subject. In that manner, a synergistic result between therapy and stimulation is achieved and provides an improvement to the region that receives stimulation and physical therapy over either alone. Alternatively, the therapy can be guided to focus on target regions that respond worse to the stimulation than other regions of the subject. In that manner, the therapy can be used to augment those target regions to improve their response to the stimulation.

Methods of the invention can be used for any neurological condition, such as neurodegenerative conditions or neuro-traumatic conditions. Examples below illustrate methods of the invention from treating patients with Parkinson's Disease and osteoarthritis (OA), but the methods are not limited to these diseases. In certain embodiments, the subject has a degenerative condition, such as Parkinson's disease. In the case of Parkinson's disease, an example of implementation involves providing stimulation that results in modulation of target regions such as the subject's upper arms above the elbow, the subject's lower arms below the elbow, and the subject's hands. In certain embodiments, the hands are more responsive to the stimulation than either the upper arms or the lower arms. In those embodiments, the focused physical therapy may be provided to the hands. Alternatively, the focused physical therapy may be provided to the upper arms, the lower arms, or a combination thereof. In the case of Parkinson's disease, another example of implementation involves providing stimulation that results in modulation of target regions such as the subject's upper legs above the knee, the subject's lower legs below the knee, and the subject's ankles. In certain embodiments, the ankles are more responsive to the stimulation than either the upper legs or the lower legs. In those embodiments, the focused physical therapy may be provided to the ankles. Alternatively, the focused physical therapy may be provided to the upper legs, the lower legs, or a combination thereof.

In certain embodiments, the subject has a condition such as OA which can result in a state of joint dysfunction and/or chronic pain which can limit patient use of joints and motor function. In the case of OA, an example of implementation involves providing stimulation that results in modulation of target regions such as the subject's upper arms above the elbow, the subject's lower arms below the elbow, and the subject's hands. In certain embodiments, the hands are more responsive to the stimulation than either the upper arms or the lower arms. In those embodiments, the focused physical therapy may be provided to the hands. Alternatively, the focused physical therapy may be provided to the upper arms, the lower arms, or a combination thereof. In the case of OA, another example of implementation involves providing stimulation that results in modulation of target regions such as the subject's upper legs above the knee, the subject's lower legs below the knee, and the subject's ankles. In certain embodiments, the ankles are more responsive to the stimulation than either the upper legs or the lower legs. In those embodiments, the focused physical therapy may be provided to the ankles. Alternatively, the focused physical therapy may be provided to the upper legs, the lower legs, or a combination thereof.

Any type of stimulation known in the art may be used with methods of the invention, and the stimulation may be provided in any clinically acceptable manner. For example, the stimulation may be provided invasively and/or noninvasively. Preferably, the stimulation is provided in a noninvasive manner. For example, electrodes may be configured to be applied to the specified tissue, tissues, or adjacent tissues. As one alternative, the electric source may be implanted inside the specified tissue, tissues, or adjacent tissues.

Exemplary types of stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). Other exemplary types include implant methods such as deep brain stimulation (DBS), microstimulation, spinal cord stimulation (SCS), and vagal nerve stimulation (VNS). In other embodiments, the stimulation source may work in part through the alteration of the nervous tissue electromagnetic properties, where stimulation occurs from an electric source capable of generating an electric field across a region of tissue and a means for altering the permittivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

In particular embodiments, the stimulation is Electrosonic Stimulation (ESStim), as described herein and in U.S. patent application number 2008/0046053, the content of which is incorporated by reference herein in its entirety. The components of the tissue stimulation method according to the present disclosure are fabricated from materials suitable for a variety of medical applications, such as, for example, polymerics, gels, films, and/or metals, depending on the particular application and/or preference. Semi-rigid and rigid polymerics are contemplated for fabrication, as well as resilient materials, such as molded medical grade polyurethane, as well as flexible or malleable materials. The motors, gearing, electronics, power components, electrodes, and transducers of the method may be fabricated from those suitable for a variety of medical applications. The method according to the present disclosure may also include circuit boards, circuitry, processor components, etc. for computerized control. One skilled in the art, however, will realize that other materials and fabrication methods suitable for assembly and manufacture, in accordance with the present disclosure, also would be appropriate.

The following discussion includes a description of the components and exemplary methods for generating currents in biological tissues in accordance with the principles of the present disclosure. Alternate embodiments are also disclosed. Reference will now be made in detail to the exemplary embodiments of the present disclosure illustrated in the accompanying figures wherein like reference numerals indicate the similar parts throughout the figures.

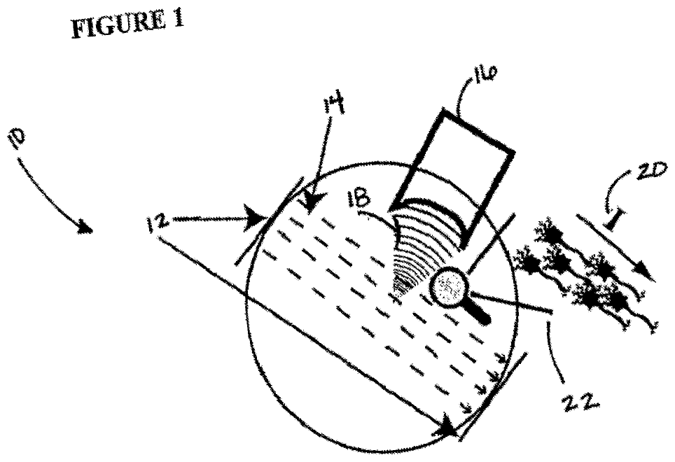

Turning now to FIG. 1, which illustrates an exemplary embodiment of an apparatus 10 to alter currents, e.g., amplify, focus, alter direction, and/or attenuate in the presence of an applied electric field or applied current source by the combined application of a mechanical field within a biological material to stimulate the biological cells and/or tissue in accordance with the present disclosure. For example, the apparatus 10 illustrated in FIG. 1 according to the present disclosure may be applied to the area of neural stimulation. An initial source electric field 14 results in a current in the tissue. The electric field 14 is created by an electric source, current or voltage source. As described in further detail below, the permittivity of the tissue is altered relative to the electric field, for example by a mechanical field, thereby generating an additional displacement current.

Electrodes 12 are applied to the scalp and generate a low magnitude electric field 14 over a large brain region. While electrodes 12 are used and applied to the scalp in this exemplary embodiment, it is envisioned that the electrodes may be applied to a number of different areas on the body including areas around the scalp. It is also envisioned that one electrode may be placed proximal to the tissue being stimulated and the other distant, such as one electrode on the scalp and one on the thorax. It is further envisioned that electric source could be mono-polar with just a single electrode, or multi-polar with multiple electrodes. Similarly, the electric source may be applied to tissue via any medically acceptable medium. It is also envisioned that means could be used where the electric source does not need to be in direct contact with the tissue, such as for example, inductive magnetic sources where the entire tissue region is placed within a large solenoid generating magnetic fields or near a coil generating magnetic fields, where the magnetic fields induce electric currents in the tissue.

The electric source may be direct current (DC) or alternating current (AC) and may be applied inside or outside the tissue of interest. Additionally, the source may be time varying. Similarly, the source may be pulsed and may be comprised of time varying pulse forms. The source may be an impulse. Also, the source according to the present disclosure may be intermittent.

A mechanical source such as an ultrasound source 16 is applied on the scalp and provides concentrated acoustic energy 18, i.e., mechanical field to a focused region of neural tissue, affecting a smaller number of neurons 22 than affected by the electric field 14, by the mechanical field 18 altering the tissue permittivity relative to the applied electric field 14, and thereby generating the altered current 20. The mechanical source may be any acoustic source such as an ultrasound device. Generally, such device may be a device composed of electromechanical transducers capable of converting an electrical signal to mechanical energy such as those containing piezoelectric materials, a device composed of electromechanical transducers capable of converting an electrical signal to mechanical energy such as those in an acoustic speaker that implement electromagnets, a device in which the mechanical source is coupled to a separate mechanical apparatus that drives the system, or any similar device capable of converting chemical, plasma, electrical, nuclear, or thermal energy to mechanical energy and generating a mechanical field.

Furthermore, the mechanical field could be generated via an ultrasound transducer that could be used for imaging tissue. The mechanical field may be coupled to tissue via a bridging medium, such as a container of saline to assist in the focusing or through gels and/or pastes which alter the acoustic impedance between the mechanical source and the tissue. The mechanical field may be time varying, pulsed, an impulse, or may be comprised of time varying pulse forms. It is envisioned that the mechanical source may be applied inside or outside of the tissue of interest. There are no limitations as to the frequencies that can be applied via the mechanical source, however, exemplary mechanical field frequencies range from the sub kHZ to 1000 s of MHz. Additionally, multiple transducers providing multiple mechanical fields with similar or differing frequencies, and/or similar or different mechanical field waveforms may be used—such as in an array of sources like those used in focused ultrasound arrays. Similarly, multiple varied electric fields could also be applied. The combined fields, electric and mechanical, may be controlled intermittently to cause specific patterns of spiking activity or alterations in neural excitability. For example, the device may produce a periodic signal at a fixed frequency, or high frequency signals at a pulsed frequency to cause stimulation at pulse frequencies shown to be effective in treating numerous pathologies. Such stimulation waveforms may be those implemented in rapid or theta burst TMS treatments, deep brain stimulation treatments, epidural brain stimulation treatments, spinal cord stimulation treatments, or for peripheral electrical stimulation nerve treatments. The ultrasound source may be placed at any location relative to the electrode locations, i.e., within, on top of, below, or outside the same location as the electrodes as long as components of the electric field and mechanical field are in the same region. The locations of the sources should be relative to each other such that the fields intersect relative to the tissue and cells to be stimulated, or to direct the current alteration relative to the cellular components being stimulated.

The apparatus and method according to the present disclosure generates capacitive currents via permittivity alterations, which can be significant in magnitude, especially in the presence of low frequency applied electric fields. Tissue permittivities in biological tissues are much higher than most other non-biological materials, especially for low frequency applied electric fields where the penetration depths of electric fields are highest. This is because the permittivity is inversely related to the frequency of the applied electric field, such that the tissue permittivity magnitude is higher with lower frequencies. For example, for electric field frequencies below 100,000 Hz, brain tissue has permittivity magnitudes as high as or greater than 10\hat{ }8 (100,000,000) times the permittivity of free space (8.854*10\hat{ }−12 farad per meter), and as such, minimal local perturbations of the relative magnitude can lead to significant displacement current generation. As the frequency of the electric field increases, the relative permittivity decreases by orders of magnitude, dropping to magnitudes of approximately 10\hat{ }3 times the permittivity of free space (8.854*10\hat{ }−12 farad per meter) for electric field frequencies of approximately 100,000 Hz. Additionally, by not being constrained to higher electric field frequencies, the method according to the present disclosure is an advantageous method for stimulating biological tissue due to lowered penetration depth limitations and thus lowered field strength requirements. Additionally, because displacement currents are generated in the area of the permittivity change, focusing can be accomplished via the ultrasound alone. For example, to generate capacitive currents via a permittivity perturbation relative to an applied electric field as described above, broad DC or a low frequency electric source field well below the cellular stimulation threshold is applied to a brain region but stimulation effects are locally focused in a smaller region by altering the tissue permittivity in the focused region of a mechanical field generated by a mechanical source such as an ultrasound source. This could be done noninvasively with the electrodes and the ultrasound device both placed on the scalp surface such that the fields penetrate the tissue surrounding the brain region and intersect in the targeted brain location, or with one or both of the electrodes and/or the ultrasound device implanted below the scalp surface (in the brain or any of the surrounding tissue) such that the fields intersect in the targeted region.

A displacement current is generated by the modification of the permittivity in the presence of the sub threshold electric field and provides a stimulatory signal. In addition to the main permittivity change that occurs in the tissues, which is responsible for stimulation (i.e., the generation of the altered currents for stimulation), a conductivity change could also occur in the tissue, which secondarily alters the ohmic component of the currents. In a further embodiment, the displacement current generation and altered ohmic current components may combine for stimulation. Generally, tissue conductivities vary slightly as a function of the applied electric field frequency over the DC to 100,000 Hz frequency range, but not to the same degree as the permittivities, and increase with the increasing frequency of the applied electric field. Additionally in biological tissues, unlike other materials, the conductivity and permittivity do not show a simple one-to-one relationship as a function of the applied electric field frequency. The permittivity ranges are as discussed above.

Although the process described may be accomplished at any frequency of the applied electric field, the method in an exemplary embodiment is applied with lower frequency applied electric fields due to the fact the permittivity magnitudes of tissues, as high as or greater than 10\hat{ }8 times the permittivity of free space, and the electric field penetration depths are highest for low frequency applied electric fields. Higher frequency applied electric fields may be less desirable as they will require greater radiation power to penetrate the tissue and/or a more pronounced mechanical source for permittivity alteration to achieve the same relative tissue permittivity change, i.e., at higher applied electric field frequencies the permittivity of the tissue is lower and as such would need a greater overall perturbation to have the same overall change in permittivity of a tissue as at a lower frequency. Applied electric field frequencies in the range of DC to approximately 100,000 Hz frequencies are advantageous due to the high tissue permittivity in this frequency band and the high penetration depth for biological tissues at these frequencies. In this band, tissues are within the so called ‘alpha dispersion band’ where relative tissue permittivity magnitudes are maximally elevated (i.e., as high as or greater than 10\hat{ }8 times the permittivity of free space). Frequencies above approximately 100,000 to 1,000,000 Hz for the applied electric fields are still applicable for the method described in generating displacement currents for the stimulation of biologic cells and tissue, however, both the tissue permittivity and penetration depth are limited for biological tissues in this band compared to the previous band but displacement currents of sufficient magnitude can still be generated for some applications. In this range, the magnitude of the applied electric field will likely need to be increased, or the method used to alter the permittivity relative to the applied electric field increased to bring about a greater permittivity change, relative to the tissue's permittivity magnitude for the applied electric field frequency. Additionally, due to potential safety concerns for some applications, it may be necessary to limit the time of application of the fields or to pulse the fields, as opposed to the continuous application that is possible in the prior band. For tissues or applications where the safety concerns preclude the technique in deeper tissues, the technique could still be applied in more superficial applications in a noninvasive manner or via an invasive method. Higher frequency applied electric fields, above 1,000,000 to 100,000,000 Hz, could be used in generating displacement currents for the stimulation of biologic cells and tissue. However, this would require a more sufficient permittivity alteration or electromagnetic radiation, and as such is less than ideal in terms of safety than the earlier bands. For frequencies of the applied electric field above 100,000,000 Hz, biologic cell and tissue stimulation may still be possible, but may be limited for specialized applications that require less significant displacement currents.

The focus of the electric and mechanical fields to generate an altered current according to the present disclosure may be directed to various structures within the brain or nervous system including but not limited to dorsal lateral prefrontal cortex, any component of the basal ganglia, nucleus accumbens, gastric nuclei, brainstem, thalamus, inferior colliculus, superior colliculus, periaqueductal gray, primary motor cortex, pre motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, cortical structures, spinal cord, nerve roots, sensory organs, and peripheral nerves.

The focused tissue may be selected such that a wide variety of pathologies may be treated. Such pathologies that may be treated include but are not limited to Multiple Sclerosis, Amyotrophic Lateral Sclerosis (ALS), Alzheimer's Disease, Dystonia, Tics, Spinal Cord Injury, Traumatic Brain Injury (TBI), Drug Craving, Food Craving, Alcohol Craving, Nicotine Craving, Stuttering, Tinnitus, Spasticity, Parkinson's Disease, Parkinsonism (aka., Parkinsonianism which includes Parkinson's Plus disorders such as Progressive Supranuclear Palsy, Multiple Systems Atrophy, and/or Corticobasal syndrome, and/or Cortical-basal ganglionic degeneration), tauopathies, synucleinopathies, Dementia with Lewy bodies, Obsessions, Depression, ADHD, Schizophrenia, Bipolar Disorder, Acute Mania, Catonia, Post-Traumatic Stress Disorder, Autism, Chronic Pain Syndrome, Phantom Limb Pain, Epilepsy, Stroke, Auditory Hallucinations, Movement Disorders (e.g., Parkinson's Disease, neuromuscular disorders (ALS, muscular dystrophies)), Neurodegenerative Disorders, Pain Disorders, Metabolic Disorders, Addictive Disorders, Psychiatric Disorders, neuropathies (e.g., such as caused by diabetes, vitamin deficiency, repetitive stress, systemic diseases, autoimmune disorders, inherited disorders, Charcot-Marie-Tooth disease), Nerve Injury or pathology (e.g., traumatic (TBI) nerve injury, metabolic and/or vascular (diabetes) neural injury, degenerative neural pathologies, infection based neural injuries (bacterial (e.g., bacterial meningitis) and/or viral (e.g., polio or viral meningitis))), and/or Sensory Disorders. Furthermore, stimulation (such as for example electric and mechanical fields to generate an altered current) may be focused on specific brain or neural structures to enact procedures including sensory augmentation, sensory alteration, anesthesia induction and maintenance, brain mapping, epileptic mapping, neural atrophy reduction, neuroprosthetic interaction or control with nervous system, stroke and traumatic injury neurorehabilitation, bladder control, assisting breathing, cardiac pacing, muscle stimulation (directly and/or through neural connections such as for example for use in upper motor neuron pathology, spinal cord injury, and/or muscle atrophy), and treatment of pain syndromes, such as those caused by migraine, neuropathies, and low-back pain; or internal visceral diseases, such as chronic pancreatitis or cancer. The methods herein could be expanded to any form of arthritis, impingement disorders, overuse injuries, entrapment disorders, spinal disorders and/or any muscle, skeletal, or connective tissue disorder which leads to chronic pain, central sensitization of the pain signals, neuropathology, and/or an inflammatory response.

In the focused region of tissue to which the mechanical fields are delivered, the excitability of individual neurons can be heightened to the point that the neurons can be stimulated by the combined fields, or be affected such as to cause or amplify the alteration of the neural excitability caused by the altered currents, either through an increase or decrease in the excitability of the neurons. This alteration of neural excitability can last past the duration of stimulation and thus be used as a basis to provide lasting treatment. Additionally, the combined fields can be provided in multiple, but separate sessions to have a summed, or carry-over effect, on the excitability of the cells and tissue. The combined fields can be provided prior to another form of stimulation, to prime the tissue making it more or less susceptible to alternate, follow-up forms of stimulation. Furthermore, the combined fields can be provided after an alternate form of stimulation, where the alternate form of stimulation is used to prime the tissue to make it more or less susceptible to the form of stimulation disclosed herein. Furthermore, the combined fields could be applied for a chronic period of time.

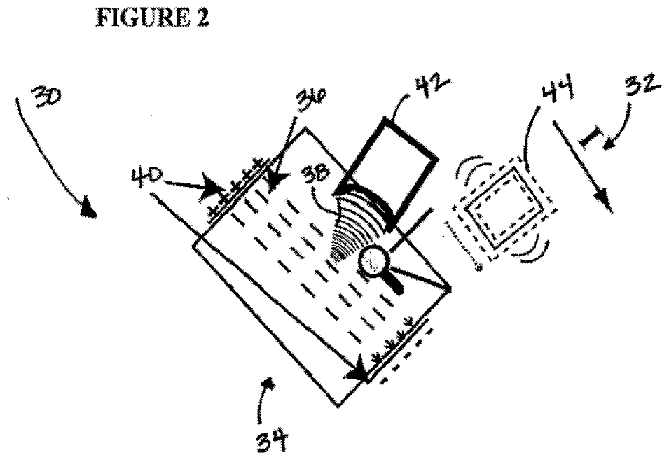

FIG. 2 illustrates a set up 30 to perform a method for generating an altered current with a newly generated displacement current 32 for stimulation in biologic tissue 34 through the combined effects of an electric field 36 and a mechanical field 38. A tissue or composite of tissues 34 is placed adjacent to the anode and cathode of an electric source 40 which generates an electric field 36. The electric field 36 is combined with a mechanical, e.g., ultrasound field 38 which can be focused on the tissue 34 and generated via an ultrasound transducer 42. In a sub-region of tissue 44 where the mechanical field 38 is focused and intersects with the electric field 36, a displacement current 32 is generated. By vibrating and/or mechanically perturbing the sub-region of tissue 44, the permittivity of the tissue 44 can be altered relative to the applied electric field 36 to generate a displacement current 32 in addition to the current that would be present due to the source electric field 36 and altered due to conductivity changes in the tissue caused by the mechanical perturbation.

By providing the mechanical field 38 to the sub region of tissue 44, the permittivity can be altered within the electric field 36 by either new elements of the sub region of tissue 44 vibrating in and out of the electric field such that the continuum permittivity of the tissue is changed relative to the electric field 36, or that the bulk properties of the sub region of tissue 44 and the permittivity, or tissue capacitance, change due to the mechanical perturbation. An example of altering the permittivity within the electric field can occur when a cell membrane and extra-cellular fluid, both of different permittivities, are altered in position relative to the electric field by the mechanical field. This movement of tissues of different permittivity relative to the electric field will generate a new displacement current. The tissues could have permittivity values as high as or greater than 10\hat{ }8 times the permittivity of free space, differ by orders of magnitude, and/or have anisotropic properties such that the tissue itself demonstrates a different permittivity magnitude depending on the relative direction of the applied electric field. An example of altering permittivity of the bulk tissue occurs where the relative permittivity constant of the bulk tissue is directly altered by mechanical perturbation in the presence of an electric field. The mechanical source, i.e., ultrasound source may be placed at any location relative to the electrode locations, i.e., within or outside the same location as the electrodes, as long as components of the electric field and mechanical field are in the same region.

Tissue permittivities can be altered relative to the applied electric fields via a number of methods. Mechanical techniques can be used to either alter the bulk tissue permittivity relative to an applied electric field or move tissue components of differing permittivities relative to an applied electric field. There are no specific limitations to the frequency of the mechanical field that is applied as previously discussed, however, exemplary frequencies range from the sub kHZ to 1000 s of MHz. A second electromagnetic field could be applied to the tissue, at a different frequency than the initial frequency of the applied electromagnetic field, such that it alters the tissue permittivity at the frequency dependent point of the initially applied electric field. An optical signal could also be focused on the tissues to alter the permittivity of the tissue relative to an applied electric field. A chemical agent or thermal field could also be applied to the tissues to alter the permittivity of the tissue relative to an applied electric field. These methods could also be used in combination to alter the tissue permittivity relative to an applied electric field via invasive or noninvasive methods.

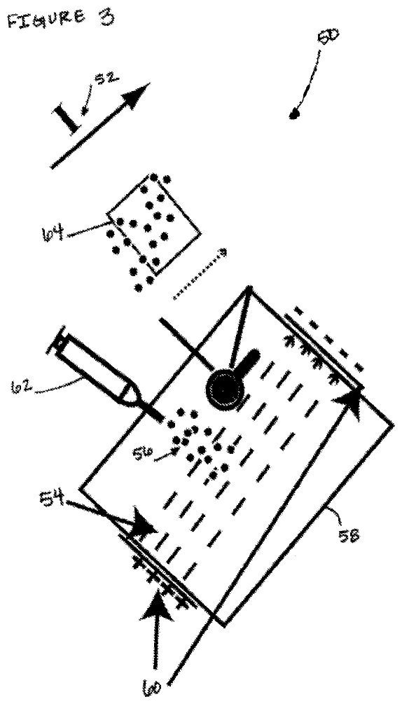

For example, FIG. 3 shows a set-up 50 for generating an altered current with a newly generated displacement current 52 through the combined effects of an electric field 54 and a chemical agent 56. A tissue or composite of tissues 58 is placed within an electric source 60 which generates an electric field 54 and combined with chemical source 62 which releases a chemical agent 56 that can be focused on the tissue 58. In the area that the chemical agent 56 is released in the tissue 64, the electric field 54 transects the sub region of tissue 64, and the chemical agent 56 reacts with the sub region of tissue 64 to alter the tissue's relative permittivity relative to the applied electric field 54. This generates a displacement current 52 in addition to the current that would be present due to the source electric field 54. The chemical agent 56 may be any agent which can react with the tissue or cellular components of the tissue 64 to alter its permittivity relative to the electric field 54. This may be by a thermoreactive process to raise or lower the tissue 64 temperature or through a chemical reaction which alters the distribution of ions in the cellular and extra-cellular media, for instance, along ionic double layers at cell walls in the tissue 64. Similarly, the conformation of proteins and other charged components within the tissue 64 could be altered such that the permittivity of the tissue is altered relative to the low frequency electric field 54. The agent could also be any agent that adapts the permanent dipole moments of any molecules or compounds in the tissue 64, temporarily or permanently relative to the low frequency electric field 54. The chemical reaction driven by the chemical agent 56 must work rapidly enough such that the permittivity of the tissue is quickly altered in the presence of the electric field 54 in order to generate the displacement current 52. The reaction may also be such as to fluctuate the permittivity, such that as the permittivity continues to change displacement currents continue to be generated. In addition to the main permittivity change that occurs in the tissues, a conductivity change could also occur in the tissue, which secondarily alters the ohmic component of the currents. A biological agent may be used in place of, or in addition to, the chemical agent 56. This embodiment may have particular application for focused drug delivery where an additional chemical or biological agent is included to assist in therapy of the tissue, or where the altered current could drive an additional electrochemical reaction for therapy. For example, this could be used in areas such as focused gene therapy or focused chemotherapy.

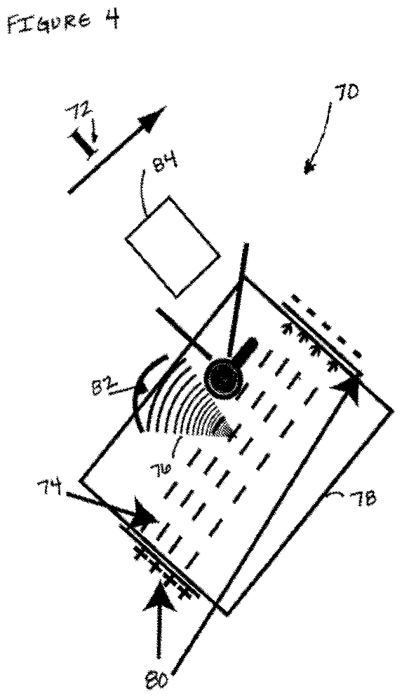

Another example is shown in FIG. 4, which illustrates a set up 70 for applying a method for generating an altered current with a newly generated displacement current 72 through the combined effects of a low frequency electric field 74 and an electromagnetic radiation field 76. A tissue or composite of tissues 78 is placed within a low frequency electric field 74 which is generated by an electric source 80 and combined with radiation source 82 which generates a radiation field 76 that can be focused on the tissue 78. In the area that the radiation field 76 is focused in the tissue 78, the electric field 74 transects the sub component of tissue 84, where the radiation field 76 interacts with the sub component of tissue 84 to alter the tissue's relative permittivity relative to the applied electric field 74, and as such generates a displacement current 72 in addition to the current that would be present due to the source electric field 74 or the radiation source field 76 alone. The electromagnetic radiation field 76 could, for example, interact with the tissue 84 by altering its temperature through ohmic processes, alter the distribution of ions in the cellular and extra-cellular media for instance along ionic double layers along cell walls through the electric forces acting on the ions, or alter the conformation of proteins and other charged components within the tissue through the electric forces such that the permittivity of the tissue is altered relative to the low frequency electric field 74. Furthermore, the electromagnetic field 76, could interact with the tissue 84 by moving components of the tissue via electrorestrictive forces, as would be seen in anisotropic tissues, to alter the continuum permittivity of the tissue relative to the low frequency electric field 74. In addition to the main permittivity change that occurs in the tissues, a conductivity change could also occur in the tissue, which secondarily alters the ohmic component of the currents.

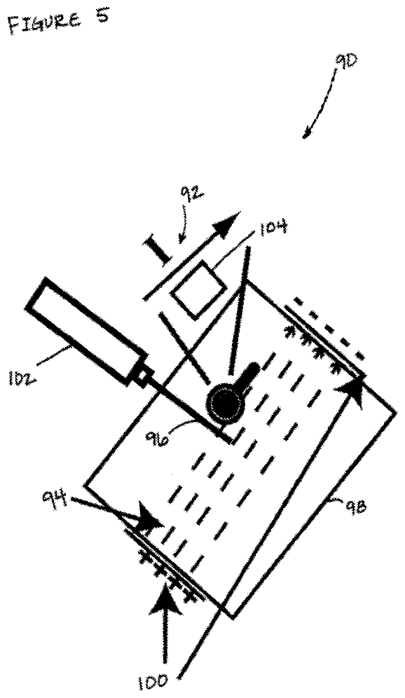

FIG. 5 shows a set-up 90 for applying a method for generating an altered current with a newly generated displacement current 92 through the combined effects of an electric field 94 and an optical beam 96. A tissue or composite of tissues 98 is placed within electric field 94 generated by an electric source 100 and combined with optical source 102 which generates optical beam 96 that can be focused on the tissue 98. In the area that the optical beam 96 is focused on the tissue, the electric field 94 transects the sub component of tissue 104, where the optical beam 96 reacts with the tissue to alter the tissue's relative permittivity relative to the applied electric field 94, and as such generates a displacement current 92 in addition to the current that would be present due to the source electric field 94. The optical beam 96 could, for example, interact with the tissue by altering its temperature through photothermal effects and/or particle excitation, alter the distribution of ions in the cellular and extra-cellular media for instance along ionic double layers along cell walls by exciting the movement of ions optically, ionizing the tissue via laser tissue-interactions, or alter the conformation of proteins and other charged components within the tissue such that the permittivity of the tissue is altered relative to the low frequency electric field 94. In addition to the main permittivity change that occurs in the tissues, a conductivity change could also occur in the tissue, which secondarily alters the ohmic component of the currents.

In another embodiment, a thermal source to alter the permittivity of the tissue may be used. In such embodiments, a thermal source such as a heating probe, a cooling probe, or a hybrid probe may be placed external or internal to the tissue to be stimulated. A thermal source may alter the permittivity of the tissue through the direct permittivity dependence of tissue temperature, mechanical expansion of tissues in response to temperature changes, or by mechanical forces that arise due to altered particle and ionic agitation in response to the temperature alteration such that permittivity of the tissue is altered relative to an applied electric field. In addition to the main permittivity change that occurs in the tissues, a conductivity change could also occur in the tissue, which secondarily alters the ohmic component of the currents. This embodiment may be useful for stimulation in the presence of an acute injury to the tissue where the thermal source could be used to additionally assist in the treatment of the tissue injury, for example with a traumatic brain injury or an infarct in any organ such as the heart. The tissue could be cooled or heated at the same time stimulation is provided to reduce the impact of an injury.

The methods discussed herein can further make use of the feedback and imaging methods described in Wagner et al. (U.S. patent application publication number 2011/0275927), the content of which is incorporated by reference herein in its entirety.

This brain stimulation technology and methods described herein can be integrated with any form of physical therapy for the treatment and/or management of symptoms of diseases (e.g., movement disorders, brain injury, osteoarthritis). Any type of physical therapy, occupational therapy, behavioral therapy, speech therapy, neuro-rehabilitative, and/or therapeutic methods such as for example those described in Dreeben-Irimia (Physical Therapy Clinical Handbook For Ptas Jones & Bartlett Learning; 2 edition; 2012); Martin et al. (Neurologic Interventions for Physical Therapy, 2e: Saunders; 2 edition; 2006); Shankman et al. (Fundamental Orthopedic Management for the Physical Therapist Assistant, 3e: Mosby; 3 edition 2010); O'Sullivan et al. (Physical Rehabilitation (O'Sullivan, Physical Rehabilitation): F. A. Davis Company; 6 edition; 2013); Shumway-Cook et al. (Motor Control: Translating Research into Clinical Practice: LWW; Fourth, North American Edition edition; 2011. 656 p.); Umphred et al. (Neurological Rehabilitation, 6e (Umphreds Neurological Rehabilitation): Mosby; 6 edition; 2012); Guccione et al. (Geriatric Physical Therapy, 3e Mosby; 3 edition; 2011); Cook (Orthopedic Manual Therapy (2nd Edition) Prentice Hall; 2 edition; 2011); Schell et al. (Occupational Therapy LWW; Twelfth, North American Edition edition; 2013); Radomski et al. (Occupational Therapy for Physical Dysfunction: LWW; Seventh, North American Edition edition; 2013); Shamliyan et al. (Physical Therapy Interventions for Knee Pain Secondary to Osteoarthritis. Rockville (Md.) 2012); Fernandes et al. (EULAR recommendations for the non-pharmacological core management of hip and knee osteoarthritis. Ann Rheum Dis. 2013; 72(7):1125-35. doi: 10.1136/annrheumdis-2012-202745. PubMed PMID: 23595142); Uthman et al., Exercise for lower limb osteoarthritis: systematic review incorporating trial sequential analysis and network meta-analysis. BMJ. 2013; 347:f5555. doi: 10.1136/bmj.f5555. PubMed PMID: 24055922; PubMed Central PMCID: PMC3779121); and Wang et al., (Physical therapy interventions for knee pain secondary to osteoarthritis: a systematic review. Ann Intern Med. 2012; 157(9):632-44. doi: 10.7326/0003-4819-157-9-201211060-00007. PubMed PMID: 23128863). The content of each of these references is incorporated by reference herein in its entirety. The techniques described in those references can be used as the physical therapy methods described herein and used with neuro stimulation.

Additional physical therapy methods that can be used in methods of the invention are described in the following:
Coggrave M, Norton C, Cody J D. Management of faecal incontinence and constipation in adults with central neurological diseases. Cochrane Database Syst Rev. 2014; 1:CD002115. doi: 10.1002/14651858.CD002115.pub5. PubMed PMID: 24420006;
Tomlinson C L, Patel S, Meek C, Herd C P, Clarke C E, Stowe R, et al. Physiotherapy versus placebo or no intervention in Parkinson's disease. Cochrane Database Syst Rev. 2013; 9:CD002817. doi: 10.1002/14651858.CD002817.pub4. PubMed PMID: 24018704;
Lima L O, Scianni A, Rodrigues-de-Paula F. Progressive resistance exercise improves strength and physical performance in people with mild to moderate Parkinson's disease: a systematic review. Journal of physiotherapy. 2013; 59(1):7-13. doi: 10.1016/S1836-9553(13)70141-3. PubMed PMID: 23419910;
Hesse S, Schattat N, Mehrholz J, Werner C. Evidence of end-effector based gait machines in gait rehabilitation after CNS lesion. NeuroRehabilitation. 2013; 33(1):77-84. doi: 10.3233/NRE-130930. PubMed PMID: 23949037;
Sitja Rabert M, Rigau Comas D, Fort Vanmeerhaeghe A, Santoyo Medina C, Roque i Figuls M, Romero-Rodriguez D, et al. Whole-body vibration training for patients with neurodegenerative disease. Cochrane Database Syst Rev. 2012; 2:CD009097. doi: 10.1002/14651858.CD009097.pub2. PubMed PMID: 22336858;
Payne C, Wiffen P J, Martin S. Interventions for fatigue and weight loss in adults with advanced progressive illness. Cochrane Database Syst Rev. 2012; 1:CD008427. doi: 10.1002/14651858.CD008427.pub2. PubMed PMID: 22258985;
de Dreu M J, van der Wilk A S, Poppe E, Kwakkel G, van Wegen E E. Rehabilitation, exercise therapy and music in patients with Parkinson's disease: a meta-analysis of the effects of music-based movement therapy on walking ability, balance and quality of life. Parkinsonism Relat Disord. 2012; 18 Suppl 1:S114-9. doi: 10.1016/S1353-8020(11)70036-0. PubMed PMID: 22166406;
Archer T, Fredriksson A, Schutz E, Kostrzewa R M. Influence of physical exercise on neuroimmunological functioning and health: aging and stress. Neurotoxicity research. 2011; 20(1):69-83. doi: 10.1007/s12640-010-9224-9. PubMed PMID: 20953749;
Ahlskog J E. Does vigorous exercise have a neuroprotective effect in Parkinson disease? Neurology. 2011; 77(3):288-94. doi: 10.1212/WNL.0b013e318225ab66. PubMed PMID: 21768599; PubMed Central PMCID: PMC3136051;
Xu Q, Park Y, Huang X, Hollenbeck A, Blair A, Schatzkin A, et al. Physical activities and future risk of Parkinson disease. Neurology. 2010; 75(4):341-8. doi: 10.1212/WNL.0b013e3181ea1597. PubMed PMID: 20660864; PubMed Central PMCID: PMC2918886;
Goodwin V A, Richards S H, Taylor R S, Taylor A H, Campbell J L. The effectiveness of exercise interventions for people with Parkinson's disease: a systematic review and meta-analysis. Mov Disord. 2008; 23(5):631-40. doi: 10.1002/mds.21922. PubMed PMID: 18181210;
Chard S E. Community neurorehabilitation: a synthesis of current evidence and future research directions. NeuroRx: the journal of the American Society for Experimental NeuroTherapeutics. 2006; 3(4):525-34. doi: 10.1016/j.nurx.2006.07.002. PubMed PMID: 17012066; PubMed Central PMCID: PMC3593402; Deane K H, Ellis-Hill C, Jones D, Whurr R, Ben-Shlomo Y, Playford E D, et al. Systematic review of paramedical therapies for Parkinson's disease. Mov Disord. 2002; 17(5):984-91. doi: 10.1002/mds.10197. PubMed PMID: 12360547;
de Goede C J, Keus S H, Kwakkel G, Wagenaar R C. The effects of physical therapy in Parkinson's disease: a research synthesis. Arch Phys Med Rehabil. 2001; 82(4):509-15. PubMed PMID: 11295012; and
Sanjay Salgado, Nori Williams, Rima Kotian, and Miran Salgado. An Evidence-Based Exercise Regimen for Patients with Mild to Moderate Parkinson's Disease. Brain Sci. 2013, 3, 87-100; doi:10.3390/brainsci3010087.

The content of each of these references is incorporated by reference herein in its entirety. The techniques described in those references can be used as the physical therapy methods described herein and used with neurostimulation.

Other types of physical therapy can be used, such as dance therapy, robotic gait training (Picelli et al., Parkinsonism & Related Disorders. 2012 18(8):990-3) and/or any other therapy delivered with robotics, pool therapy (i.e., in a swimming pool), and/or any other water-based therapy. Therapy types can for example be constraint-induced movement therapy, neurodevelopmental therapy, Bobath method, Brunnstrom approach, Rood approach, sensory integration technique, animal assistance based therapy, mobilization therapy, virtual reality based robotic therapy, virtual reality based therapy, mental imagery methods, motor imagery methods, mirror, therapy, activities of daily living (ADL) functional training, joint taping (e.g., with athletic tape supporting a joint), any physical therapy method described in (Veerbeek et al., PloS one. 2014; 9(2):e87987. doi: 10.1371/journal.pone.0087987. PubMed PMID: 24505342; PubMed Central PMCID: PMC3913786) including therapeutic positioning arm, reflex-inhibiting immobilization, air-splints, supportive techniques or devices for the prevention or treatment of glenohumeral subluxation and/or hemiplegic shoulder pain, bilateral arm training, electromyography biofeedback, trunk restraint, therapeutic massage, chiropractic manipulations, gait and balance training techniques, sitting balance training, sit to stand training, standing balance training (with and without biofeedback), balance training during various activities, balance board training, body-weight supported treadmill training, electromechanical-assisted gait training, speed dependent treadmill training, over-ground walking, rhythmic gait cueing, community walking, virtual reality mobility training, circuit class training, caregiver-mediated exercises, orthosis for walking, and/or electromyography biofeedback. Along with the neurostimulation, any therapy method described herein can be implemented individually or in combination with any other therapy methods (and/or other therapy methods detailed herein), in any order and/or combination and/or before, during, after, and/or synchronized with neurostimulation in any combination.

Physical therapy can include an activity (or activities) and/or intervention(s) that engage a specific brain region(s) that is stimulated and/or connected to area(s) of stimulation (e.g., the basal ganglia, motor cortex, supplementary motor cortex, cerebellum, thalamus), such as for example to further improve the impact of stimulation and therapy (for example stimulation might improve distal function of the arm, and physical therapy might further improve distal function of the arm, and by coupling the therapies together the distal arm function would be more effectively improved). Physical therapy can include an activity (or activities) and/or intervention(s) that engage a specific brain region(s) that is not directly or indirectly stimulated by a stimulation source, such as for example to couple the effects of therapy with those of stimulation (for example transcranial stimulation can be used to improve distal function of the arm, and specific physical therapy improves proximal function of the arm, and by coupling the therapies together the arm function would be improved as a whole; or for example transcranial stimulation can be used improve ankle function, and specific physical therapy improves knee function, and by coupling the therapies together the leg function and/or gait would be improved). Therapy can include an activity (or activities) and/or intervention(s) that engage a specific brain region(s) that is stimulated by stimulation and/or be connected to an area(s) of stimulation and be maximally affected. Therapy can include an activity (or activities) and/or intervention(s) that engage a specific brain region(s) that is stimulated by stimulation and/or connected to an area of stimulation and be more or less affected than another area (for example, a transcranial stimulation can be given that is determined to be maximally effective on elbow flexion and extension and is less effective on the shoulder and wrist function, then following the assessment a physical therapy could be given focused on wrist movements and a second therapy given focused on shoulder therapy).

Physical therapy can include an activity (or activities) and/or intervention(s) that engage a specific body region(s) (e.g., arm, proximal arm, distal arm, leg, ankle, trunk, elbow joint, fingertip, lliopsoas, quadriceps femoris, popliteus fascia, 2^{nd} rib, humerus, muscles, bones, cartilage, connective tissue, vascular tissue, endocrine tissue, organs, fascia, etc.) and/or a function(s) (e.g., arm flexion and/or extension, proximal arm flexion (e.g., elbow) compared to distal arm extension (e.g., wrist), arm flexion, arm extension, wrist flexion, wrist extension, wrist pronation, wrist supination etc.) that is stimulated by stimulation and/or connected to an area(s) of stimulation (e.g., the basal ganglia, motor cortex, supplementary motor cortex, cerebellum, thalamus). The body regions and/or functions may for example be any regions and/or functions taken from Grant's Atlas of Anatomy by Anne Agur and Ming Less (10^{th} edition, Lippincott, Williams, and Wilkins); Gray's Anatomy: The Unabridged Running Press Edition Of The American Classic by Henry Gray, T. Pickering Pick and Robert Howden (May 22, 1974); Atlas of Human Anatomy, Professional Edition (5th edition) (Netter Basic Science) by Frank H. Netter M D (May 17, 2010); Robbins and Cotran Pathologic Basis of Disease, Professional Edition: Expert Consult—Online and Print, 9e (Robbins Pathology) by Vinay Kumar, Abul K. Abbas and Jon C. Aster (Jul. 9, 2014); Robbins Pathologic Basis of Disease, 6e (Robbins Pathology) by Vinay Kumar and Abul K. Abbas (Jan. 15, 1999); Eckert Animal Physiology: Mechanisms and Adaptations by David Randall, Warren Burggren, Kathleen French and Roger Eckert (February 1997); Biomechanics of Sport and Exercise, 2nd Edition by Peter M. McGinnis (Nov. 1, 2004); Physiology of Sport and Exercise with Web Study Guide, 5th Edition by W. Larry Kenney, Jack H. Wilmore and David L. Costill (Nov. 15, 2011); and Kinematics of Human Motion by Vladimir Zatsiorsky (Sep. 9, 1997), the content of each of these references is incorporated by reference herein in its entirety. The body region(s) may be selected in any combination or order. Other functions and/or targets can also be impacted by stimulation, directly or indirectly, which could be used to improve therapy such as vascular function (such as having a differential effect on improving blood flow to particular body regions to alter functional performance), inflammatory response (such as having a differential effect on inflammation in body regions to alter functional performance), pain function, motor function, proprioceptive function (such as having a differential effect on body regions' proprioceptive sense to alter functional performance), position awareness, cognitive function, sensory function, auditory function, visual function, visuo-spatial function, vibration processing, balance function, internal temperature regulation, metabolic function, physiological function, fatigue, and/or coordination. The functional effect(s) may also be selected in any order or combination. Functional effects can be secondary to another functional effect (e.g., motor function improvement may be secondary to another primary improvement (such as for example vascular and/or pain improvements)), and this can be used in the therapy tuning. Therapy can include an activity (or activities) and/or intervention(s) that engage a specific body region(s) and/or function(s) that is stimulated by stimulation and/or connected to an area(s) of stimulation and be more or less affected than another area. Therapy can include an activity (or activities) and/or intervention(s) that engage a specific body region(s) and/or function(s) that is the site(s) of stimulation and/or connected to an area(s) of stimulation and be maximally affected.

Physical therapy can be provided before, after, or during stimulation for any duration of time. Therapy can include an activity (or activities) that increase neurotrophic factors or other metabolic agents that have a therapeutic effect such as for example brain derived neurotrophic factor (BDNF). Therapy can include an activity (or activities) that release an endogenous opioid(s) such as for example endorphins, endomorphins, dynomorphins, and/or ekephalins. Therapy can include an activity (or activities) that modulate the release, absorption, and/or generation of a neurotransmitter(s) and/or a neurotransmitter precursor(s). Therapy can include an exercise(s) or physical activity (or activities) (with or without additional equipment) such as cardiovascular exercise, aerobics, yoga, gymnastics, treadmill activities and/or other cardiovascular machine training (e.g., elliptical machine, climbing machine, stair machine, rowing machine, arm pedal machine, stationary cycling machine, etc.), swimming, running, walking, climbing, jumping rope, weight lifting, elastic band training, balance ball training, skiing, surfing, skating, manual therapy exercises, balance training, coordination training, manual therapy exercises with a partner, martial arts (e.g., Tai Chi (Hackney et al., Gait Posture. 2008 28(3):456-60)), cycling, rowing, stretching, exercising with assist devices, and/or exercising with exercise equipment. All of these exercises (and therapies described herein) can be performed alone, with a partner and/or guide, and/or any assistance device.

Physical therapy can be provided first to establish a functional baseline, i.e., to characterize an individual's response to therapy. Stimulation can be provided first to establish an individual's response to stimulation. A functional baseline of an individual can also be determined and used as part of the method (e.g., a baseline based on a metric to be evaluated and used for tuning of the therapeutic regimen). The baseline can be based on the metrics evaluated via stimulation and/or therapy to determine their therapeutic impact and/or be independent of the metrics being evaluated for either. The baseline can be determined before any intervention is given or before any new interventions are given and/or as a function of patients' current therapies (such as for example when developing adjunctive therapeutic regimens). For example, if therapy and stimulation were being provided to improve the speed at which a patient moves their arm in a flexion and extension task of the elbow and the opening and closing of the hand, one can first establish a functional baseline prior to any intervention of the speed at which the person moves their arm through the task. One can then assess how stimulation and/or therapy impacts the performance of the task (in any order) and adjust the parameters of the interventions for the best therapeutic effect. One can also assess the effects of the therapy and/or stimulation individually and/or as a group, and adjust the other interventions accordingly. For example, in a task that includes elbow flexion and extension and hand opening and closing, a patient could respond to stimulation where hand opening and closing was maximally effected by stimulation, and thereby a physical therapy regimen could be designed around this response to stimulation to further improve the hand function (and be provided with stimulation, after stimulation, before stimulation, and/or synchronized with the therapy (e.g., at the same or different sessions)). Or similarly in a task that includes knee flexion and extension and ankle flexion and extension a patient can respond to stimulation where ankle flexion and extension was maximally effected by stimulation, and thereby a physical therapy regimen can be designed and implemented around this response to stimulation to further improve the ankle function (and be provided with stimulation, after stimulation, before stimulation, and/or synchronized with the therapy (e.g., at the same or different sessions)). This process can involve tuning either of (or both of) the interventions to specific functions, where the effects of stimulation and/or therapy can be assessed relative to each other (and/or relative to baseline evaluations) and be adjusted to maximally improve the patient relative to the desired outcome. For example, in a task that includes general upper limb movement, an individual could respond to stimulation where proximal arm movement speed was improved, and to a physical therapy regimen where proximal arm coordination was improved, one could adjust the order, magnitude, and duration of the interventions relative to each other to improve the patient's functional outcome (Herein, we use the term proximal and distal relevant to the upper limb described here to mean proximal for elbow and distal for wrist, whereby the terms proximal and distal are used relative to the trunk for the limbs, and/or further relative to the nerve roots which innervate the limbs; this same concept can be applied to other limbs, body trunk, core, etc. Similarly, the idea of axial vs distal effects can also be implemented. Additionally, the proximal/distal and axial/distal motor control concept can also used herein, wherein stimulation and therapy can be tuned around the different aspects of motor control. For example, stimulation and/or therapy can be tuned relative to motor control mechanisms, for instance stimulation therapy can have a differential effect on distal vs axial motor control mechanisms, and the identification of one mechanism or another being dominate following an intervention can be implemented relative to the other therapy being given). Stimulation and/or therapy can also be provided and/or tuned relative to a particular control mechanism being implemented by the brain and/or system being treated. Stimulation and/or therapy can also be provided and/or tuned relative to particular functional characteristics of a functional activity being treated. For example, stimulation and/or therapy can be tuned relative to the speed at which a patient is moving, for example providing different stimulation and/or therapy parameters for when a patient is extending their arm quickly compared to slowly. Furthermore, stimulation and/or therapy can be tuned to particular functional characteristics of an activity, such as for example footstep size and/or speed and/or other parameters of gait, for particular subsets of an activity, such as for example turning or walking up a flight of stairs. Furthermore, one can optimize activities of a therapeutic regimen relative to the stimulation and/or therapy being given, such as for example if a patient was undergoing a particular stimulation and physical therapy regimen where part of the physical activities required a particular treadmill activity with the patient, one could tune the speed and/or angle of the treadmill relative to the other interventions.

In some embodiments, stimulation and/or therapy may be provided and/or tuned with a therapeutic pharmacologic agent regimen and/or a single agent. For instance with Parkinson's Disease (PD), a patient could be given stimulation and/or therapy tuned around the patient being in the ‘on’ period of levodopa treatment (i.e., Although levodopa is an effective pharmacologic treatment for Parkinson's disease, there can be variability in an individual's response to treatment—so-called “motor fluctuations.” The fluctuating response to levodopa can be broadly described in “on” and “off” periods. During an “on” period, a person can move with relative ease often with reduced tremor and stiffness. “Off” periods describe those times when a person has greater difficulty with movement). See Connolly et al. (JAMA. 2014 Apr. 23-30; 311(16):1670-83. doi: 10.1001/jama.2014.3654), the content of which is incorporated by reference herein in its entirety. Stimulation and/or therapy can be given at any time with the drug regimen that is correlated with maximizing efficacy. For instance with PD, it may be shown that the maximum therapeutic effect of stimulation is attained by giving patients stimulation at the onset of their “On” period and that subsequent therapy is giving during the middle of their “On” period (and for example other functions might best respond during the “off” period). Similarly, with OA it could be demonstrated that stimulation could provide maximum therapeutic effect while a person is in a period where a pain medication's therapy is waning, but that the subsequent therapy is maximum at the onset of the effects of another dose of the pain medication. Any combination of stimulation and/or therapy around the pharmacologic regimen for maximum efficiency can be delivered.

Stimulation and/or therapy may be provided around asymmetries of disease states in pathology and/or function (for example, with Parkinson's Disease (PD) the disease is often asymmetrical in presentation from left to right side of a patient, whereby stimulation can be tuned to a patients' more effected brain hemisphere (or differentially across the hemispheres based on the disease state) and/or therapy could be tuned to the more effected side of the disability. For example, with PD, a patient might have a right-sided brain dominant disease, and the patient is in disease state where bradykinesia is expressed in most detrimentally in the contralateral left arm, and less detrimentally in the right arm. In such a patient, stimulation could be tuned to provide more stimulation therapy to the right hemisphere and subsequently a differential physical therapy focused on bradykinesia treatment focused on the more effected arm could be implemented, such as for example in diseases like PD, stroke, and cerebral palsy. One can also assess the effects of the therapy and/or therapies and/or stimulation(s) individually and/or as a group relative to a functional baseline assessment and/or relative to each other (and/or other therapies and/or stimulation if given as a group), and/or look at correlations between different effects (such as a differential effect between elbow flexion, elbow extension, ankle flexion, and/or ankle extension compared to baseline, and/or at different evaluation points (at different times or therapy and/or stimulation sessions), between stimulation(s), between therapy, between therapies, and/or between the different groups and/or components of the groups (for example between a group of stimulations and a group of therapies) and adjust the other intervention(s) accordingly (where adjustments in therapy can for example be made in altering the dose of therapy (e.g., duration, number, intensity, dates or times between therapy sessions given, etc.), location of therapy (e.g., elbow vs wrist, etc.), and/or stopping or substituting the therapy type and/or where adjustments in stimulation can for example be made to altering the dose of stimulation, the location of stimulation, and/or stopping or substituting the stimulation type). Furthermore, this tuning of stimulation and therapy can be implemented around the design of algorithms that analyze the trade-off in functional effects across the different methodologies, for example one can analyze the differential effects of stimulation and therapy on elbow flexion and extension and design the appropriate therapeutic regimen relative a to a patient's individual deficiencies.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Electrosonic Stimulation for Treating Parkinson's Disease

Electrosonic Stimulation is an improved noninvasive modality that overcomes the limitations of other noninvasive technologies by combining independently controlled electromagnetic and ultrasonic fields. The combined fields focus and boost neurostimulation currents via tuned electromechanical coupling in neural tissue. Past Electrosonic Stimulation studies demonstrated a significantly improved duration and magnitude of stimulation effect compared to other dose-matched noninvasive stimulation modalities in electrophysiology, metabolic, and behavioral studies. Given the advantages of this technique over other noninvasive options (e.g., transcranial magnetic stimulation (TMS) and transcranial direct current stimulation (Tdcs; Wagner et al., Annu Rev Biomed Eng. 2007. PubMed PMID: 17444810; Wagner et al., Cortex. 2008. Epub 2008/11/26. doi: S0010-9452(08)00231-1 [pii] 10.1016/j.cortex.2008.10.002. PubMed PMID: 19027896; and Brunoni et al., Brain Stimulation. 2011. Epub 2011/11/01. doi: 10.1016/j.brs.2011.03.002. PubMed PMID: 22037126; PubMed Central PMCID: PMC3270156.), we proposed to test whether Electrosonic Stimulation would also induce significant therapeutic effects in Parkinson's disease (PD) patients.

In the phase I portion of this study, we conducted a single-center, double-blinded, placebo controlled, randomized study in order to investigate the effects of Electrosonic Stimulation in PD patients. We specifically focused on evaluating Electrosonic Stimulation Safety (electrophysiology, cognitive, and neurological safety markers) and Motor Effects (Unified Parkinson's Disease Rating Scale (UPDRS), a bradykinesia test, and a walking ability/gait test) in PD patients who received Electrosonic Stimulation provided over the primary motor cortex (M1) for 10 days, 20 minutes/day (12 Active, 12 SHAM, results from 22 patients form the basis of this report, 11 SHAM and 11 Active). As proposed, all patients were provided stimulation and evaluated in the ‘On’ state.

We have provided stimulation to patient completion in 20 patients at the time this report was generated, with 4 patients ongoing stimulation and evaluation. Below we provide further details of the safety criteria reviewed during this study.

No serious adverse events occurred during the 215 stimulations sessions that transpired so far in the course of this study, nor were any reported in the subsequent patient follow-up visits.

Patient neurological exams revealed no additional signs or symptoms in the patients following stimulation (in neither the active nor SHAM conditions, the exam was completed by a blinded physician).

On the first day of stimulation and at the end of each stimulation week, EEG recordings were taken to monitor brain activity using a 64 channel EEG for 20 mins. No demonstration of seizure activity related to stimulation (defined by epileptiform discharges) nor any other pathological EEG activity (such as activity slowing, spikes, and synchronized activity) were observed following stimulation (in neither the active nor SHAM conditions, the EEG evaluator was blinded).

We assessed a full battery of neurocognitive metrics, including tests for: Scales for Outcomes in PD-Cognitive (SCOPA-Cog), working memory, peg board exam, and Visual Analog Scale metrics (depression, anxiety, sleep and stress). The Scales for Outcomes in PD-Cognitive (SCOPA-Cog) instrument is recommended by the NIH as part of its CDE program for PD research. The testing is based on measures of cognitive performance in Memory and learning, Attention, Executive functions, Visuo-spatial functions, and memory. During the 4-Choice Reaction Time test patients responded via a key press whenever a target figure appeared on screen, using the correct corresponding response key. The figure remains on screen until the correct button is pressed. This instrument has been used in past brain stimulation studies before as it measures general attention. As used in our previous PD study, Working Memory tests were conducted where subjects were shown a semi-randomized group of 11 letters (from A to J). Each letter remained on a computer screen for 300 ms. A new letter was shown every 2 s. Subjects responded via key press whenever the letter presented was already presented 3 letters previously. We also analyzed VAS measures (depression, anxiety, sleep and stress).

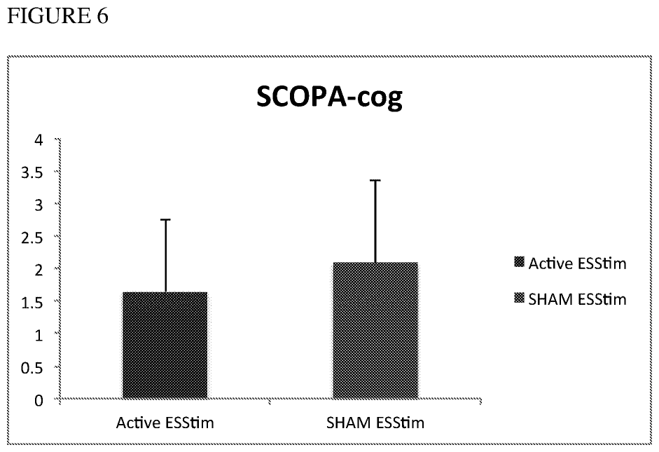

Our primary analysis was based on comparing the relative change in measures between the last day of stimulation (day 10) and the baseline measures in the Active Electrosonic Stimulation and SHAM conditions. We demonstrated non-significant differences in SCOPA-cog tests between Active and SHAM stimulations, here with a 1.63 and 2.09 score increase (p=0.32, t-test) between baseline and the last day of stimulation—see FIG. 6, showing the mean change across the 20 patients who completed stimulation.

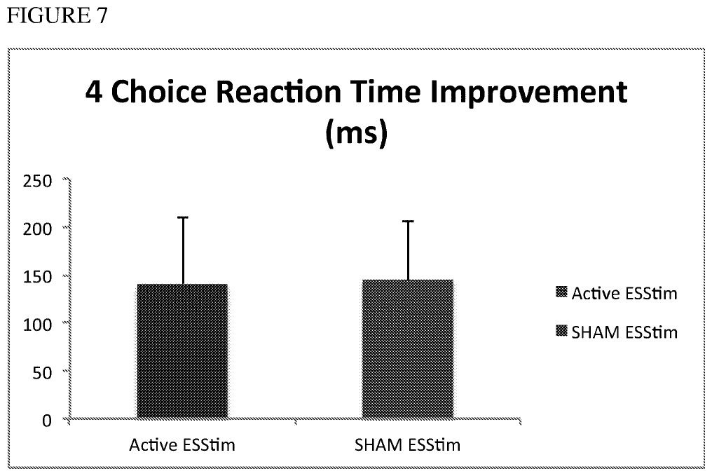

We demonstrated a non-significant change in 4 choice-reaction times between Active and SHAM stimulations, here with a 140.21 ms improvement and a 145.04 ms improvement (p=0.48, t-test) between baseline and the last day of stimulation—see FIG. 7.

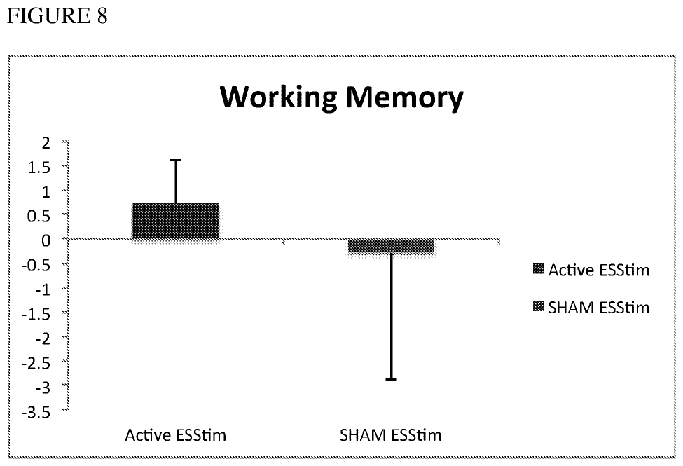

We demonstrated a non-significant change in working memory between Active and SHAM stimulations, here with a 0.72 increase and a 0.27 score decrease (p=0.39, t-test) between baseline and the last day of stimulation—see FIG. 8.

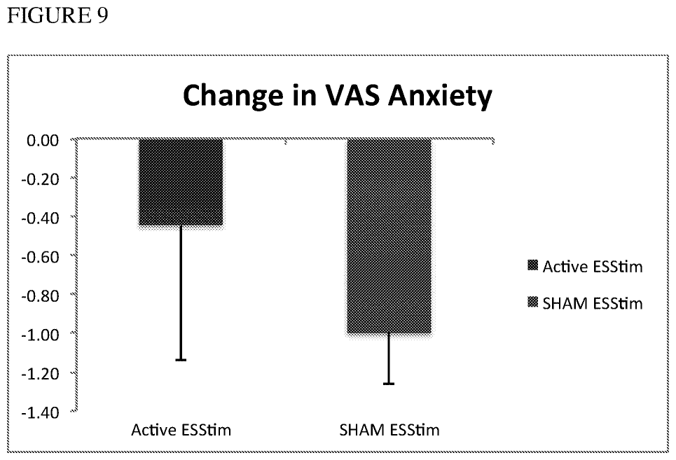

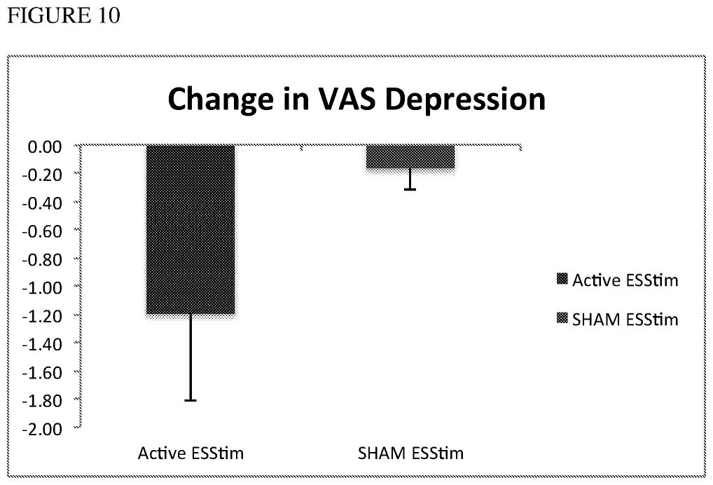

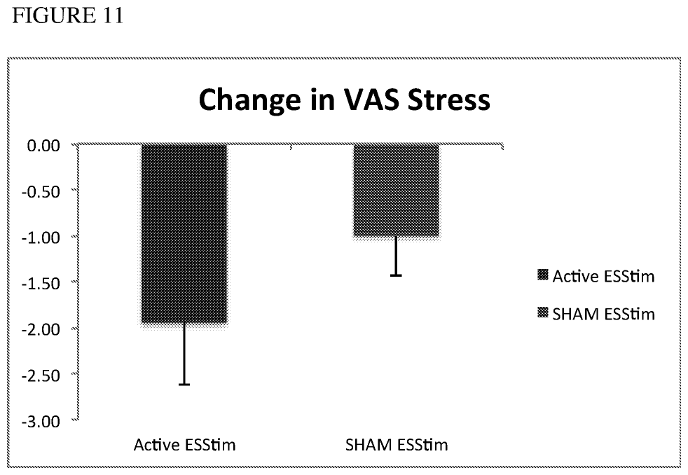

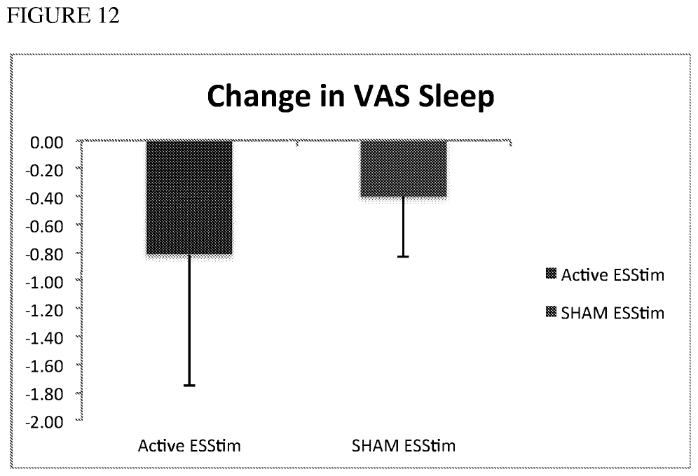

We also analyzed VAS measures (depression, anxiety, sleep and stress). We demonstrated non-significant change in VAS Anxiety between Active and SHAM stimulations, here with a 0.44 decrease and a 1.00 score decrease (p=0.76, t-test) between baseline and the last day of stimulation—see FIG. 9 (note—decrease in scores indicates that the patient is less anxious). We demonstrated a non-significant changes in VAS Depression between Active and SHAM stimulations, here with a 1.19 decrease and a 0.20 score decrease (p=0.072, t-test) between baseline and the last day of stimulation—see FIG. 10 (note—decrease in scores indicate that the patient is less depressed). We demonstrated an insignificant changes in VAS Stress between Active and SHAM stimulations, with a 1.94 decrease and a 1.00 score decrease (p=0.12, t-test) between baseline and the last day of stimulation—see FIG. 11 (note—decrease in scores indicate that the patient is experiencing less stress). We demonstrated an insignificant changes in VAS Sleep between Active and SHAM stimulations, with a 0.81 decrease and a 0.41 score decrease (p=0.34, t-test) between baseline and the last day of stimulation—see FIG. 12 (note-decrease in scores indicate that the patient is experiencing less levels of sleepiness (i.e., patients with lower scores would indicate that they had slept better the prior nights)).

### Example 2: Electrosonic Stimulation for VAS Depression

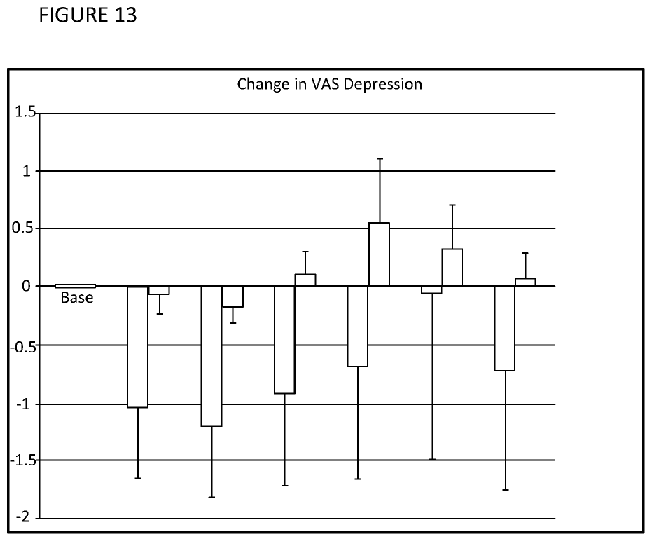

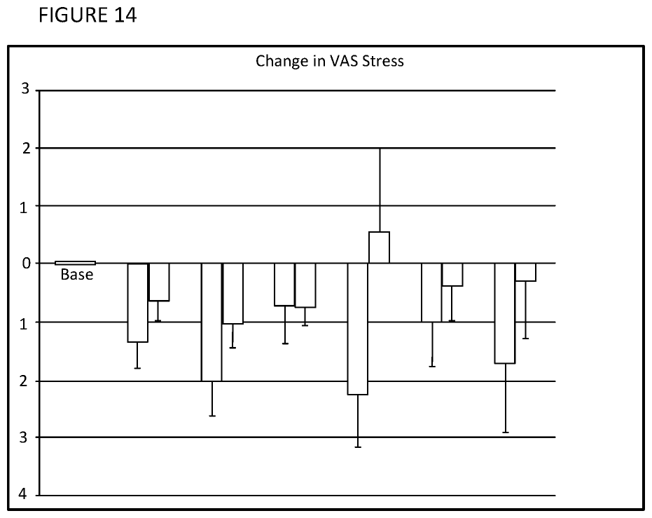

Other forms of noninvasive stimulation have been used to treat depression, including tDCS and TMS (primarily through stimulating the dorsal lateral prefrontal cortex (DLPFC)). However, a number of studies have demonstrated the improvement of mood in patients following motor cortex stimulation (i.e., the location of the Electrosonic Stimulation transducer). Additionally, the VAS Depression test (described above) approached significance while comparing Active and SHAM conditions (p=0.072). Given that we have acquired additional data from these patients (from measurements at the baseline (Base), following the first stimulation session (Post 1), following the 5 stimulation session (Post 5), following the 10th stimulation session (Post 10), at the first follow-up visit 1 week post stimulation (FU1), at the second follow-up visit 2 weeks post stimulation (FU2), at the third follow-up visit 1 month post stimulation (FU3), and at the last follow-up visit (FU2)) we analyzed the data via a 2 way-ANOVA analyzing the effects of stimulation on VAS Depression (Dependent: Change in VAS Depression/Independent: Visit, Stimulation Type). The 2 way ANOVA demonstrated a significant effect for Stimulation Type (p=0.013). We also ran a 2 way-ANOVA analyzing the effects of stimulation on VAS Stress (Dependent: Change in VAS Stress/Independent: Visit, Stimulation Type), demonstrating a significant effect of stimulation type (p=0.014). See FIGS. 13-14.

### Example 3: Electrosonic Stimulation Reduces Symptoms of Parkinson's Disease

We have provided stimulation to patient completion in 20 patients at the time this report was generated, with 4 patients ongoing treatment and analysis. This efficacy data is focused on the 20 patients who have completed all of their stimulation sessions.

As our primary endpoints, we tested:
Walking Time/Gait: Times were measured for a patient to walk 10 m. This was done three times and averaged.
We assessed the patient UPDRS scores (we assessed Parts I-IV, which we will review herein—but we will primarily focus on the motor section results (Part III)).
Bradykinesia: We assessed movement in the arms and hands by asking patients to perform a sequence of motions 10 times (hand opening and closing, extension and flexion of the elbow, and squeezing a ball and opening again).

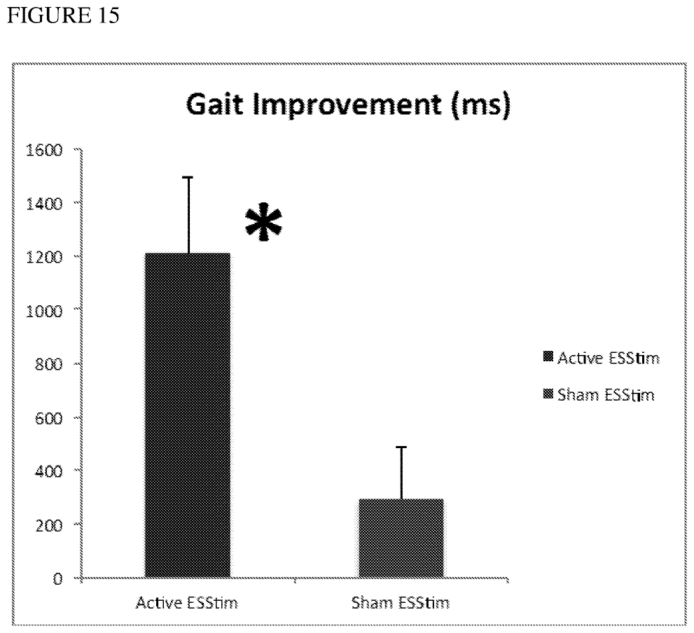

We designed our study to compare Walking Time improvements between the Active Electrosonic Stimulation and SHAM Electrosonic Stimulation conditions on the last day of stimulation (i.e. the difference in times to walk ten meters as measured at baseline and following the last day of stimulation (i.e., day 10)). We demonstrated a significant improvement in walking times comparing Active and SHAM stimulations, with 1212 ms vs 294 ms improvements following the last stimulation session compared to baseline (p=0.0063, t-test). This represents a 312.98% change relative to SHAM. See FIG. 15.

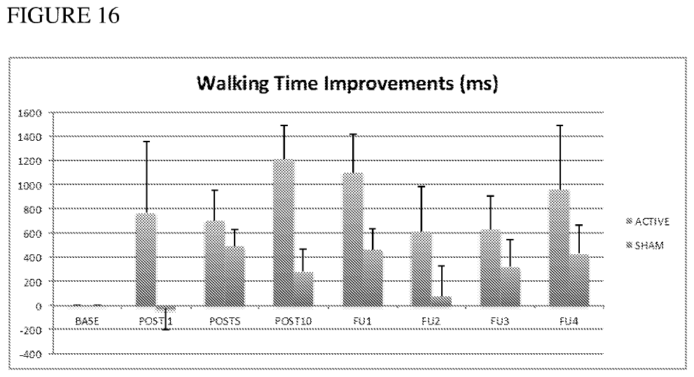

Given that we have acquired additional data from these patients (from measurements at the baseline (Base), following the first stimulation session (Post 1), following the 5 stimulation session (Post 5), following the 10th stimulation session (Post 10), at the first follow-up visit 1 week post stimulation (FU1), at the second follow-up visit 2 weeks post stimulation (FU2), at the third follow-up visit 1 month post stimulation (FU3), and at the last follow-up visit (FU2)) we analyzed the data via 2 way-ANOVA (Dependent: Improvement in Walking Time/Independent: Visit, Stimulation Type) and demonstrated a significant effect for Stimulation Type (p=0.0029). See FIG. 16.

Secondarily, we also performed a paired analysis where patient were paired based on their baseline UPDRS scores and ran a 3 way-ANOVA (Dependent: Improvement in Walking Time for Baseline Independent: Visit, Stimulation Type, and Patient Pairing (paired based on baseline performance)) and demonstrated a significant effect for Stimulation Type (p=0.01), Visit (p=0.024), and Pairing (p=0.003) and significant interaction effects for Pairing and Stimulation Type (p=0.004). This interaction effect between stimulation type and pairing suggests that Electrosonic Stimulation may be particularly effective, relative to sham stimulation, in a subpopulation of patients, based on their baseline scores before stimulation. Specifically, we found that patients with smaller baseline scores were more likely to demonstrate a large improvement in walking time with Electrosonic Stimulation, relative to sham stimulation. This finding may allow us to identify patient for whom Electrosonic Stimulation therapy is most likely to be immediately effective (or to allow us to determine patients who can be targeted for a focused type of physical therapy and stimulation therapy).

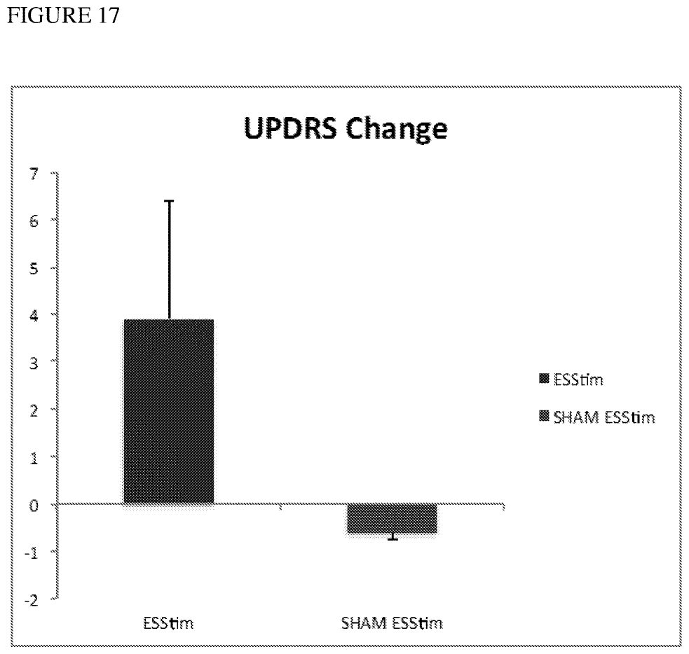

Electrosonic Stimulation efficacy was also investigated via the Unified Parkinson's Disease Rating Scale (UPDRS) Part I-IV (total UPDRS). We demonstrated a 4.5 total point improvement comparing Active and SHAM stimulations following the last stimulation session compared to baseline, with a trend toward significance (p=0.058, unpaired t-test of differences)—see FIG. 17 (note baseline determined at pre-simulation clinic visits). The average of our patients' baseline UPDRS was 29.7, so a 4.5 improvement in these ‘ON’ patients was clinically significant, especially given that Electrosonic Stimulation is planned as an adjunctive therapy to patients' current physical/medical therapies (51). Given the aim of this study was to change the neural excitability of motor neural targets, we also performed an analysis focusing on the motor aspects of the UPDRS. We demonstrated a 2.5 UPDRS Part III total point improvement comparing Active and SHAM differences following the last stimulation session relative to baseline. Furthermore, we analyzed separately the components of UPDRS related to rigidity and bradykinesia. This analysis showed a difference of 2.5 times (or 250%) in score differences between the two groups (SHAM and Active Electrosonic Stimulation), indicating a significantly larger improvement in the Active Electrosonic Stimulation group (p=0.04, unpaired t-test of differences) for the bradykinesia & rigidity components.

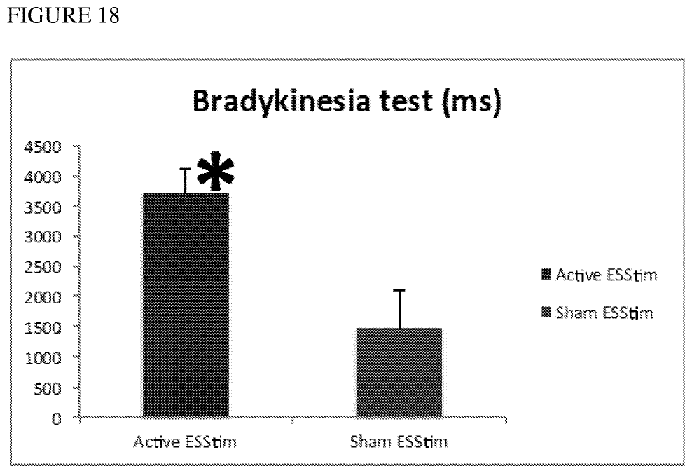

The only comparable study in the literature which compared Active and SHAM tDCS effects on UPDRS scores of patients in the ‘ON’ state, in a comparable methodology to our study, demonstrated non-significant changes on UPDRS, and in fact, they demonstrated a 1.9 points LOWER UPDRS score change in the Active tDCS group compared to SHAM stimulation following the last stimulation session (i.e., SHAM showed a greater improvement in UPDRS scores compared to tDCS) (52). No comparable TUS studies were identified (see below for a Meta-Analysis other noninvasive brain stimulation studies in relation to UPDRS scores). We designed our Bradykinesia study to compare the time to perform a set of movements (comprising ball squeezes, flexion-extension, and a hand open and closing movements). We compared the time improvements (between baseline and last day of stimulation) in the Active and SHAM Electrosonic Stimulation conditions. We demonstrated a significant improvement in the bradykinesia test times comparing Active and SHAM stimulations, with 3711 ms vs 1477.25 ms improvements following the last stimulation session compared to baseline (p=0.0033, t-test). This represents a 151.21% change relative to SHAM. See FIG. 18.

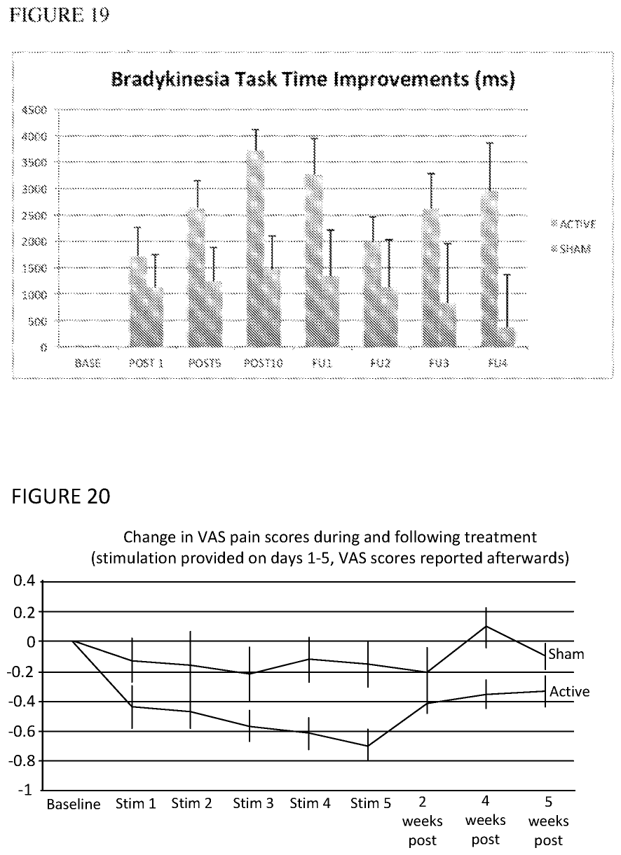

Given that we have acquired additional data from these patients (Baseline, Post 1, Post 5, Post 10, FU1, FU 2, FU 3, and FU 4), we also ran 2 way-ANOVA (Dependent: Improvement in Bradykinesia Test Time for Baseline Independent: Visit, Stimulation Type) demonstrated a significant effect for Stimulation Type (p=0.0001) and a significant effect for Visit (p=0.017). See FIG. 19. We also ran a 3 way-ANOVA (Dependent: Improvement in Bradykinesia Test Time Independent: Visit, Stimulation Type, and Patient Pairing (paired based on baseline performance)) demonstrated a significant effect for Stimulation Type (p=6.9e⁻⁴), Visit (p=1.4e⁻⁵), and Pairing (p=3.0e⁻⁷) and significant interaction effects for Pairing and Stimulation Type (p=5.7e⁻⁶). This data shows that stimulation can be coupled with focused physical therapy based on movements and tasks that respond most to the stimulation. For example, we have identified patient types who are most responsive to therapy, i.e., based on baseline UPDRS scores, and the way in which they have responded to stimulation by improving in bradykinesia tasks. We could implement a physical therapy regimen designed to improve the velocity of movement, such as exercise and/or coordination tasks focused on improving movement speed and quality (which can have their intensities adjusted relative to their baseline performances).

In a subset of 8 patients we ran a secondary analysis, analyzing which of the movements were most affected by stimulation (ball squeeze, hand-open-close, flexion-extension). In 8 patients (4 SHAM, 4 Active) matched for starting bradykinesia test performance times, we compared their relative improvements on each of the individual movements relative to baseline. In these patients we demonstrated that Active groups improved 1.2 times better than SHAM in flexion-extension movements, 1.9 times better in the hand-open and closing movements, and 2.6 times better in the ball squeezing movements. This data shows that stimulation can be coupled with focused physical therapy based on movements and tasks that respond most to the stimulation. For example, in this subset of patients, they improved more with hand tasks compared to flexion and extension tasks at the elbow. Given this, one can implement a therapeutic regimen to improve hand tasks further, such as a set of hand exercises designed to improve grasping tasks, and couple it to a stimulation regimen as necessary. Or one can provide a therapeutic regimen that provides greater therapy to the arm function relative to the hand function, in the period following stimulation, such that there is a balance in therapeutic effect across the arm and hand, whereby greater physical therapy to the arm makes up for the limited effects seen from stimulation in the hand.

### Example 4: Electrosonic Stimulation Reduces Symptoms of Osteoarthritis (OA)

Osteoarthritis (OA) of the knee is a leading cause of chronic pain and disability in the elderly. Limitations in efficacy and safety of current pharmacological therapies necessitate the development of alternative therapies. Noninvasive brain stimulation therapies have been successfully explored for the treatment of other forms of chronic pain, whereby stimulation induced changes in cortical excitability revert maladaptive plasticity associated with the perception/sensation of chronic pain. Electrosonic Stimulation is an improved noninvasive modality that overcomes limitations of other noninvasive technologies by combining independently controlled electromagnetic and ultrasonic fields. It is believed that Active Electrosonic Stimulation could be efficacious in suppressing the perception of pain relative to Sham stimulation.

We initially assessed the therapeutic effects of Electrosonic Stimulation in OA to determine whether Electrosonic Stimulation is effective in reducing pain in OA subjects with chronic pain of the knee as measured by changes in the Visual Analogue Scale (VAS) for pain and to determine whether the therapeutic effects of Electrosonic Stimulation can persist past the period of stimulation (i.e., providing offline pain relief). We also address how stimulation can be coupled with tuned physical therapy to enhance stimulation effects.

Eighteen OA patients with chronic pain of the knee were recruited and randomly assigned to an Active (n=9) or Sham (n=9) group. All patients provided informed consent as the study was performed in accordance with the Declaration of Helsinki (1964). Electrosonic Stimulation (electrical: ˜2 mA/35 cm²; sonic: ˜2.2 MHz/0.2 W/cm²) was focused on the patients' M1 for 20 minutes/day for 5 days. Pain was assessed via VAS scores taken at baseline (before any intervention was given), at the end of the stimulation visits, and at 2, 4, and 6 weeks post stimulation.

Patients that received Active Electrosonic Stimulation showed 69% reduction in VAS scores relative to baseline following the 5^{th} day of stimulation, with significant effects lasting up to 4 weeks post-stimulation (35% reduction at 4 weeks). A 2-way ANOVA of the VAS scores was significant for visit number (p<0.05) and stimulation type (p<0.001). Note that a 10% decrease is shown below as a −0.1 score relative to baseline. See FIG. 20.

Following stimulation, patients will have either a greater improvement in knee flexion compared to knee extension, a greater improvement in knee extension compared to knee flexion, or no greater improvement in either movement relative to each other related to stimulation. Patients also will have either a greater improvement in ankle flexion compared to ankle extension, a greater improvement in ankle extension compared to ankle flexion, or no greater improvement in either movement relative to each other related to stimulation. It is believed that these patients will demonstrate a permutation of response groups that can be provided tuned therapy following stimulation, focusing on the differential effects of stimulation. For example, it is believed that patients that respond with equal improvements in flexion and extension across both joints can benefit from a general strengthening, flexibility building, and aerobic exercises focused on the lower limb (particularly given that their pain perception was also reduced), however patients that show just increased flexion in the ankle and knee could have a therapy focused around the limitations in their lower limb joint extension. It is believed that with further treatment, focused stimulation and/or focused therapy can be given, such is to areas feeding different neuro-tracts to the ankle and knee accordingly (and/or to individual muscles for flexion and extension), in a differential manner relative to therapy to maximize the overall therapeutic effect. Furthermore, it is believed that given the fact that their lower limb pain suppression is increased with stimulation dose (i.e., more stimulation sessions), more intense therapeutic sessions can be provided as a function stimulation session. Furthermore, it is believed that this concept can be extended to tuning other interventions that are given in conjunction with the therapies under study, such as for example one can implement an algorithm to alter the pain medication a patient is receiving relative to the patient's response to the tuned stimulation and physical therapy. Finally, the motor function improvement (or any functional improvement) from stimulation and/or therapy may be secondary to another primary improvement, and this can be used in the therapy and/or stimulation tuning. For example stimulation can be provided which has an effect on pain to an affected joint, this could manifest in a way to improve motor function in a differential manner (and/or the pain reduction can be differential), such as for example stimulation could be provided such that general knee pain is lessened but quadriceps pain and function are less improved than hamstring pain and function, thereby one could then plan and implement for focused therapy focused on the quadriceps (and/or additional pain suppression therapy could also be provided to the muscle).

What is claimed is:

1. A method for guiding physical therapy to a subject, the method comprising:
providing a first stimulation, via one or more stimulation devices, to a subject's central nervous system to modulate one or more signals sent to or from a first target region of the subject;
providing a second stimulation, via the one or more stimulation devices, to the subject's central nervous system to modulate one or more signals sent to or from a second target region of the subject, wherein the second stimulation is of the same as the first stimulation;
comparing a first response of the first target region to the first stimulation with a second response to the second target region to the second stimulation to determine if there is a differential response among the first and second target regions to the first and second stimulation; and
providing focused physical therapy to at least one of the first and second target regions based on the differential response.

2. The method according to claim 1, wherein focused physical therapy is provided to the first target region that is more responsive to the stimulation than the second target region.

3. The method according to claim 1, wherein focused physical therapy is provided to the first target region that is less responsive to the stimulation than the second target region.

4. The method according to claim 1, further comprising altering a stimulation provided to the subject in response the subject's response to the physical therapy.

5. The method according to claim 1, wherein the subject has a degenerative condition.

6. The method according to claim 5, wherein the degenerative condition is Parkinson's disease.

7. The method according to claim 6, wherein the target regions are the subject's upper arms above the elbow, the subject's lower arms below the elbow, and the subject's hands.

8. The method according to claim 7, wherein the hands are more responsive to the stimulation than either the upper arms or the lower arms.

9. The method according to claim 8, wherein the focused physical therapy is provided to the hands.

10. The method according to claim 8, wherein the focused physical therapy is provided to a region selected from the group consisting of: the upper arms, the lower arms, and a combination thereof.

11. The method according to claim 1, wherein the signal is processed in the brain.

12. The method according to claim 1, wherein the first and second stimulations are noninvasive.

13. The method according to claim 1, wherein the first and second stimulations are selected from the group consisting of: mechanical, optical, electromagnetic, thermal, and a combination thereof.

14. The method according to claim 1, wherein the first and second stimulations occur from an electric source capable of generating an electric field across a region of tissue and a means for altering impedance of the tissue relative to the electric field is selected from the group consisting of: a chemical source, an optical source, a mechanical source, a thermal source, an electromagnetic source, and a combination thereof.

15. The method according to claim 1, wherein the first and second stimulations are provided by a combination of an electric field and a mechanical field.

16. The method according to claim 15, wherein the mechanical field is generated by an ultrasound device.

17. The method according to claim 15, wherein said electric field is pulsed.

18. The method according to claim 15, wherein said electric field is time varying.

19. The method according to claim 15, wherein the electric field is pulsed a plurality of time, and each pulse may be for a different length of time.

20. The method according to claim 9, wherein said electric field is time invariant.
